SEQUENCE LISTING (37 sequences)

SEQ ID NO: 1 - DNA, 20 bp, Artificial
  Nucleotide sequence of splice acceptor location (from 5′ boundary) of PGK-492 promoter
tcaccgacctctctccccag

SEQ ID NO: 2 - DNA, 23 bp, Artificial
  Nucleotide sequence of splice acceptor location (from 5′ boundary) of EFS (EF1? short) - 184 promoter
cgcaacgggtttgccgccagaac

SEQ ID NO: 3 - DNA, 23 bp, Artificial
  Nucleotide sequence of Promoter and spice acceptor location (from 5′ boundary) of Ubc-902 promoter
taggcttttctccgtcgcaggac

SEQ ID NO: 4 - DNA, 23 bp, Artificial
  Nucleotide sequence of splice acceptor location (from 5′ boundary) of Ubc-1134 promoter
ggtcaatatgtaattttcagtgt

SEQ ID NO: 5 - DNA, 22 bp, Artificial
  Nucleotide sequence of splice acceptor location (from 5′ boundary) of Ubc-1189 promoter
gtttttggcttttttgttagac

SEQ ID NO: 6 - DNA, 23 bp, Artificial
  Nucleotide sequence of splice acceptor location (from 5′ boundary) of SFFV-371 promoter
cccctcactcggcgcgccagtcc

SEQ ID NO: 7 - DNA, 23 bp, Artificial
  Nucleotide sequence of splice acceptor location (from 5′ boundary) of SFFV-374 promoter
gcgcgccagtcctccgacagact

SEQ ID NO: 8 - DNA, 23 bp, Artificial
  Nucleotide sequence of splice acceptor location (from 5′ boundary) of SV40-171 promoter
ccgcccctaactccgcccagttc

SEQ ID NO: 9 - DNA, 23 bp, Artificial
  Nucleotide sequence of  splice acceptor location (from 5′ boundary) of SV40-229 promoter
aattttttttatttatgcagagg

SEQ ID NO: 10 - DNA, 23 bp, Artificial
  Nucleotide sequence of splice acceptor location (from 5′ boundary) of CMV-252 promoter
gggactttcctacttggcagtac

SEQ ID NO: 11 - DNA, 23 bp, Artificial
  Nucleotide sequence of consensus sequence around active splice acceptor in PGK promoter
tcaccgacctctctccccagcct

SEQ ID NO: 12 - DNA, 22 bp, Artificial
  Nucleotide sequence of consensus sequence around active splice acceptor in modified PGK promoter
tcaccgacctctccccgagcct

SEQ ID NO: 13 - DNA, 55 bp, Artificial
  Nucleotide sequence of splice trap ST1
tccttaacgggttttttttttttttttccaggttgccgccaccatgtaaatgtag

SEQ ID NO: 14 - DNA, 54 bp, Artificial
  Nucleotide sequence of splice trap ST2
tccttaacggcccttctccctttcttccaggttgccgccaccatgtaaatgtag

SEQ ID NO: 15 - DNA, 57 bp, Artificial
  Nucleotide sequence of splice trap ST3
ttcgttgaccgaatcaccgacctctctccccagcctgccgccaccatgtaaatgtag

SEQ ID NO: 16 - DNA, 55 bp, Artificial
  Nucleotide sequence of splice trap ST4
tcagtggttcaaagtttttttcttccatttcagggccgccaccatgtaaatgtag

SEQ ID NO: 17 - DNA, 55 bp, Artificial
  Nucleotide sequence of splice trap ST5
accatgttcatgccttcttctttttcctacagctgccgccaccatgtaaatgtag

SEQ ID NO: 18 - DNA, 60 bp, Artificial
  Nucleotide sequence of splice trap ST5
aagttaagtaatagtccctctctccaagctcacttacaggccgccaccatgtaaatgtag

SEQ ID NO: 19 - DNA, 510 bp, Artificial
  Nucleotide sequence encoding modified PGK promoter in which the guanine in the active splice site is deleted.
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccca

SEQ ID NO: 20 - DNA, 509 bp, Artificial
  Nucleotide sequence encoding modified PGK promoter in which the 3′ adenine and guanine in the active splice site are deleted (PGKnoSA deleted)
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccc

SEQ ID NO: 21 - DNA, 511 bp, Human
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 22 - DNA, 7 bp, Artificial
  match to consensus for splice donor
aggtaag

SEQ ID NO: 23 - DNA, 16 bp, Artificial
  match to consensus for splice acceptor
atatatatatatcagg

SEQ ID NO: 24 - DNA, 23 bp, Artificial
  EF1?
gtttttttcttccatttcaggtg

SEQ ID NO: 25 - DNA, 34 bp, Artificial
  SV40 SEQUENCE
agttccgcccattctccgccccatggctgactaa

SEQ ID NO: 26 - DNA, 23 bp, Artificial
  Synthetic sequence
tcaccgacctctctccccagcct

SEQ ID NO: 27 - DNA, 22 bp, Artificial
  Synthetic sequence
tcaccgacctctccccgagcct

SEQ ID NO: 28 - DNA, 59 bp, Artificial
  noSA_1F
ccggagcgcacgtcggcagtcggctccctcgttgaccgaatcaccgacctctctccccg

SEQ ID NO: 29 - DNA, 59 bp, Artificial
  noSA_1R
aattcggggagagaggtcggtgattcggtcaacgagggagccgactgccgacgtgcgct

SEQ ID NO: 30 - DNA, 119 bp, Artificial
  Fig2b, top strand. Synthetic sequence
aagcctccggagcgcacgtcggcagtcggctccctcgttgaccgaatcaccgacctctctccccagcctagggaattcgccgccaccatggtgagcaagggcgaggagctgttcaccgg

SEQ ID NO: 31 - DNA, 119 bp, Artificial
  Fig2b, bottom strand Synthetic sequence
ccggtgaacagctcctcgcccttgctcaccatggtggcggcgaattccctaggctggggagagaggtcggtgattcggtcaacgagggagccgactgccgacgtgcgctccggaggctt

SEQ ID NO: 32 - protein, 11 aa, Artificial
  Fig2b, protein sequence
MVSKGEELFTG

SEQ ID NO: 33 - DNA, 111 bp, Artificial
  Fig2b, synthetic sequence
aagcctccggagcgcacgtcggcagtcggctccctcgttgaccgaatcaccgacctctctccccgaattcgccgccaccatggtgagcaagggcgaggagctgttcaccgg

SEQ ID NO: 34 - DNA, 65 bp, Artificial
  Fig4a ST1_F ST1_F
cgattccttaacgggttttttttttttttttccaggttgccgccaccatgtaaatgtagcaccgt

SEQ ID NO: 35 - DNA, 60 bp, Artificial
  Fig4a ST1_R
gtgctacatttacatggtggcggcaacctggaaaaaaaaaaaaaaaacccgttaaggaat

SEQ ID NO: 36 - DNA, 70 bp, Artificial
  Top strand shown in figure 4a - synthetic sequence
atcgattccttaacgggttttttttttttttttccaggttgccgccaccatgtaaatgtagcaccgtgtg

SEQ ID NO: 37 - DNA, 70 bp, Artificial
  Bottom strand shown in figure 4a - synthetic sequence
cacacggtgctacatttacatggtggcggcaacctggaaaaaaaaaaaaaaaacccgttaaggaatcgat

(19) United States
(12) Patent Application Publication
(10) Pub. No.: US 2023/0151388 A1
(43) Pub. Date: May 18, 2023

(54) MODIFIED VECTORS FOR PRODUCTION OF RETROVIRUS

(71) Applicant: GLAXOSMITHKLINE INTELLECTUAL PROPERTY DEVELOPMENT LIMITED, Brentford (GB)

(72) Inventor: Nathan SWEENEY, Stevenage (GB)

(21) Appl. No.: 17/916,940

(22) PCT Filed: Apr. 7, 2021
(86) PCT No.: PCT/EP2021/058576

§ 371 Date: Oct. 4, 2022

(30) Foreign Application Priority Data
Apr. 7, 2020 (GB) 2005096.9

(51) Int. Cl. C12N 15/86; A61K 48/00

(52) U.S. Cl. CPC C12N 15/86; A61K 48/005; C12N 2740/16043; C12N 2800/60; C12N 2830/42

(57) ABSTRACT

The invention relates to nucleic acid modules and also to vectors comprising such modules such as retroviral transfer vectors and BACs which show reduced production of spliced vector transcripts competent for transgene expression and wherein the modules comprise modified promoters and/or “splice traps” and also express non-endogenous transgenes such as therapeutic transgenes, and to uses and methods of production thereof.

### FIELD OF THE INVENTION

The invention relates to nucleic acid modules and also to vectors comprising such modules such as retroviral transfer vectors and BACs which show reduced production of spliced vector transcripts competent for transgene expression and wherein the modules comprise modified promoters and/or “splice traps” and also express non-endogenous transgenes such as therapeutic transgenes, and to uses and methods of production thereof.

### BACKGROUND TO THE INVENTION

In gene therapy, genetic material is delivered to endogenous cells in a subject in need of treatment or alternatively it is delivered to isolated ex vivo cells. The genetic material may introduce novel genes to the subject, or introduce additional copies of pre-existing genes, or introduce different alleles or variants of genes that are present in the subject. Viral vector systems have been proposed as an effective gene delivery method for use in gene therapy (Verma and Somia (1997) Nature 389: 239-242).

In particular, these viral vectors are based on members of the retrovirus family due to their ability to integrate their genetic payload into the host's genome. Retroviral vectors are designed to keep the essential proteins required for packaging and delivery of the retroviral genome, but any non-essential accessory proteins including those responsible for their disease profile are removed. Examples of retroviral vectors include lentiviral vectors, such as those based upon Human Immunodeficiency Virus Type 1 (HIV-1), which are widely used because they are able to integrate into non-proliferating cells.

Currently, the majority of viral vectors are produced by transient co-transfection of viral genes into a host cell line (the producer cell line). The viral genes are introduced using bacterial plasmids which exist in the host cell for only a limited period of time because the viral genes remain on the plasmids and are not integrated into the genome.

The majority of manufacturing processes from lentiviral vectors involve typically transient transfection of producer cells, requiring high quality or GMP-grade plasmids, which poses a challenge in terms of costs and production scheduling. A four plasmid system is commonly used, consisting of (i) a transfer vector plasmid which contains the therapeutic transgene and also (ii) three plasmids encoding viral sequences required to produce the vector envelope glycoprotein, structural proteins and enzymes required for reverse transcription and vector integration (gag, pol, env and rev). When present in a producer cell together, these plasmids generate the RNA and proteins required for the production of the virus particle.

Alternatively in place of use of 3-4 separate plasmids as described above the manufacturing process can involve use of nucleic acid vectors comprising a non-mammalian origin of replication and the ability to hold at least 25 kilobases (kb) of DNA, (such as for example the nucleic acid vectors provided in WO 2017/089307 including for example the bacterial artificial chromosomes (BACs) described therein and/or the vectors provided in WO 2017/089308 can also be used). Such vectors comprise a non-mammalian origin of replication and the ability to hold at least 25 kilobases (kb) of DNA, characterized in that said nucleic acid vector comprises retroviral nucleic acid sequences encoding:

gag and pol proteins, and

an env protein or a functional substitute thereof.

In addition to a (therapeutic) transgene expression cassette, the transfer vector genome must contain cis-acting viral elements such as LTRs and also a leader sequence and packaging signal. Within this leader region lies a conserved major splice donor. RNA splicing caused by the use of this major splice donor along with a downstream splice acceptor site typically generates a spliced vector transcript that does not contain the complete cis-acting sequences required for efficient genome packaging and therefore this transcript cannot be packaged efficiently. Hence while essential for retroviral genome regulation in the context of infectious virus, these spliced vector transcripts are not required and if produced might interfere with efficient vector manufacture, yet preventing their generation has proved extremely challenging since the RNA conformation around the major splice donor is sensitive to modifications.

Additionally expression of the transgene e.g. the therapeutic transgene, in the producer cells is mostly from transcripts originating at the LTR and is often undesirable due to for example cytotoxic effects on the producer cells and this also has the potential to negatively impact the manufacturing and transduction process.

Hence there exists a need for improved nucleic acid vectors such as retroviral transfer vectors expressing a transgene (e.g. a therapeutic transgene) and e.g. BACs such as the BACs described above and provided in WO 2017/089307 and also e.g. the vectors described in WO 2017/089308 which comprise all of the retroviral genes essential for retroviral vector production as well as the transgene which addresses the above, and which vectors have reduced expression of the transgene in the producer cell line.

### SUMMARY OF THE INVENTION

The present inventors have therefore addressed the disadvantages described above and have provided improved nucleic acid modules for use in nucleic acid vectors, said vectors are improved vectors with reduced production of spliced vector transcripts competent for transgene expression and hence reduced expression of the transgene in the producer cell line but without impacting expression of transgene in transduced cells. This will for example enable more consistent and efficient vector manufacture and also allow production of stable producer cell lines carrying transgenes which can be problematic to such cells when expressed.

The nucleic acid modules provided by the invention comprise: (i) a modified splice acceptor site within the promoter driving transgene expression and which modified promoter functions to reduce or eliminate production of spliced transcripts at this position and/or (ii) one or more “splice traps” to redirect splicing away from the transgene cassette as are described herein; these modifications thereby reduce or eliminate expression of the transgene in producer cells. By reducing or preventing splicing onto the transgene cassette, particularly at positions where the transgene would become the 5′ ORF which is typically the ORF that is recognised and translated into protein by ribosomes, less transcripts that are competent for transgene expression shall be produced and therefore less transgene protein shall be translated in producer cells.

The use of such improved nucleic acid vectors comprising the nucleic acid modules of the invention (such as retroviral transfer vectors or BACs) comprising promoters with modified splice acceptor sites and/or comprising the splice traps described herein therefore provides advantages in the generation of retroviral producer cell lines, use of packaging cell lines when adding transfer plasmid and improves production of retrovirus e.g. retrovirus expressing therapeutic transgenes including improving transient production.

Therefore, according to a first aspect of the invention, there is provided:

A nucleic acid module encoding at least one transgene, wherein said module comprises (a) a first promoter which is operably linked to retroviral nucleic acid sequences encoding:
(i) cis-acting retroviral elements such as LTRs,
(ii) a retrovirus leader sequence comprising a splice donor site,

and (b) a second promoter for transgene expression operably linked to said transgene and wherein said second promoter for transgene expression is modified such that it comprises (a) an attenuated splice acceptor site; and/or (b) said second promoter has a splice trap sequence positioned upstream and/or downstream of said promoter which reduces or eliminates splicing at its position.

The invention also provides a nucleic acid vector such as a plasmid (e.g. transfer plasmid encoding the genome), or a bacterial artificial chromosome (BAC) comprising a module of the invention.

Also provided by the invention are methods of making nucleic acid vectors comprising a module of the invention wherein said methods comprise obtaining a nucleic acid vector e.g. a retroviral vector (such as a transfer vector) or a BAC (such as those described in WO 2017/089307), comprising the following retroviral nucleic acid sequences:

cis-acting viral elements such as LTRs, a leader sequence and packaging signal, a promoter for transgene expression operably linked said transgene(s), and also optionally retroviral nucleic acid sequences encoding gag, pol and env proteins (or functional substitute thereof) and also a non-mammalian origin of replication and then performing the step of (i) modifying the promoter for transgene expression by deleting (wholly or partially) the splice acceptor site or modifying it e.g. by insertion, deletion or substitution mutations such that it has reduced or eliminated function as a splice acceptor, thereby reducing or eliminating splicing at this location; and or (ii) inserting into said vector a “splice trap” such that it is positioned upstream and/or downstream of the transgene or is inserted between the major splice donor and transgene cassettes transcriptional start site or positioned between the major splice donor and the site of polyadenylation.

According to a further aspect of the invention, there is provided nucleic acid vectors comprising a module as defined herein for use in a method of producing replication defective retroviral vector particles.

According to a further aspect of the invention, there is also provided a method of producing a replication defective retroviral vector particle e.g. expressing a transgene such as a therapeutic transgene, comprising the following steps:
(a) introducing a nucleic acid vector comprising a module of the present invention, such as any of the modified vectors or modified BACs as described herein into a culture of host cells e.g. mammalian host cells; and
(b) culturing the host cells under conditions in which the replication defective retroviral vector particle is produced, and
(c) isolating said replication defective retroviral vector particles from said cells.

Such replication defective retroviral vector particles isolated as described above can then be e.g. purified and/or formulated e.g. with suitable excipients for ex-vivo cell transduction or e.g. formulated e.g. with suitable pharmaceutical excipients for administration to a subject e.g. a human subject in need thereof.

According to a further aspect of the invention, there is provided replication defective retroviral vector particles that are obtainable or obtained by the methods of the invention as defined herein and uses thereof.

Also provide by the invention are pharmaceutical compositions comprising the vectors and modules and transduced cells of the invention.

According to yet a further aspect of the invention, there is provided a cell line comprising the nucleic acid vectors which comprise a module of the present invention such as the modified vectors or modified BACs as described herein which are integrated into a culture of mammalian host cells.

### DESCRIPTION OF DRAWINGS/FIGURES

FIG. 1: Shows a splice site consensus map (obtained from Brent & Guigó, 2004; Recent advances in gene structure prediction. Current Opinion in Structural Biology, 14(3), 264-272).

FIGS. 2 (a) and (b): illustrate mutagenesis of a cryptic splice acceptor site located within the human PGK promoter such that its splice acceptor activity is eliminated.

Panel a) shows the sequence of oligonucleotides that were annealed together to replace the BspE1-EcoRI fragment at the 3′ end of the PGK promoter which contains the cryptic splice acceptor site. Sequences are 5′-3′. Nucleotides designed to anneal together are in capital letters, while those that generate overhangs are in lower case. Panel b) shows an annotated alignment of the targeted region between BmodT-PGW (upper) and BmodT-PGKnoSAGW (lower).

FIGS. 3a-c: Demonstrate the impact of deleting the PGK promoter cryptic splice acceptor on transgene expression and titre a) shows fluorescence microscopy with WT PGK and PGKnoSA (3 repeats are shown). b) shows fluorescence intensity in transfected and transduced cells (WT PGK and PGKnoSA plasmid construct and lentiviral vector, respectively) and c) shows transfection efficiency and titre (WT PGK and PGKnoSA). Note that in the Figure PGKnoSA is shown as PGKΔsa (these two terms are used interchangeably). 3 data points showing 3 replicates are provided.

FIG. 4: Illustrates the design and testing of splice traps as an alternative strategy to reduce splicing from the major splice donor onto transgene and shows:

(a) ST1 (b) ST1-ST6 sequences (c) Show Splice trap impact on transgene expression

FIG. 5: Shows MaxEntScan splice acceptor scores for splice traps ST1-ST6

FIG. 6: Shows comparison of P24 titre (pg/mL) of stable pools generated using wtPGK and PGKnoSA2 BAC DNA constructs as determined by use of an ELISA assay.

FIG. 7: Shows copy number of BAC DNA modules (including transfer DNA module: HIV) in stable clones generated using wtPGK and PGKnoSA2 BAC DNA constructs as determined by digital droplet PCR.

FIG. 8: Shows P24 titre (pg/mL) of stable clones generated using PGKnoSA2 and PGKnoSA2 BAC DNA constructs as determined by use of an ELISA assay

FIG. 9: Shows Infectious titre (TU/mL) of stable clones generated using wtPGK and PGKnoSA2 BAC DNA constructs determined by transducing SupT1 cells and quantification by digital droplet PCR.

FIG. 10: shows layout of elements in the transfer vector construct of example 4

FIG. 11: PGW and ST #-PGW lentiviral transfer vector designs—shows the layout of elements present in lentiviral vectors used in example 3. ST #-refers to splice trap numbers tested.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference in their entirety.

The term “comprising” encompasses “including” or “consisting” e.g. a composition “comprising” X may consist exclusively of X or may include something additional e.g. X+Y.

The term “consisting essentially of” limits the scope of the feature to the specified materials or steps and those that do not materially affect the basic characteristic(s) of the claimed feature.

The term “consisting of” excludes the presence of any additional component(s).

The term “about” in relation to a numerical value x means, for example, x±10%, 5%, 2% or 1%.

The term “vector” or “nucleic acid vector” refers to a vehicle which is able to artificially carry foreign (i.e. exogenous) genetic material into another cell, where it can be replicated and/or expressed. Examples of vectors include non-mammalian nucleic acid vectors, such as bacterial artificial chromosomes (BACs), yeast artificial chromosomes (YACs), P1-derived artificial chromosomes (PACs), cosmids or fosmids.

Other examples of vectors include viral vectors, such as retroviral and also lentiviral vectors, which are of particular interest in the present invention.

According to a first aspect of the invention, there is provided:

A nucleic acid module encoding at least one transgene, wherein said module comprises (a) a first promoter which promoter is operably linked to retroviral nucleic acid sequences encoding:
(i) cis-acting retroviral elements such as LTRs,
(ii) a retrovirus leader sequence comprising a splice donor site,

and (b) a second promoter for transgene expression operably linked to said transgene and wherein said second promoter for transgene expression is modified such that it comprises (a) an attenuated splice acceptor site; and/or (b) said second promoter has a splice trap sequence positioned upstream and/or downstream of said promoter which reduces or eliminates splicing at its position.

The invention also provides a nucleic acid vector such as a plasmid, or a bacterial artificial chromosome (BAC) comprising a nucleic acid module of the invention.

Retroviruses:

Retroviruses are a family of viruses which contain a diploid single-stranded RNA genome. They encode a reverse transcriptase which produces DNA from the RNA genome which can then be inserted into the host cell DNA.

The invention described herein may be used to produce retroviral vector particles such as lentivirus including replication defective retroviral vector particles such as replication defective lentivirus.

The retroviral nucleic acid sequences of the present invention may be selected from or derived from any suitable retrovirus.

In one embodiment, the retrovirus is derived from, or selected from, a lentivirus, alpha-retrovirus, gamma-retrovirus or foamy-retrovirus, such as a lentivirus or gamma-retrovirus, in particular a lentivirus.

In a further embodiment, the retrovirus is a lentivirus selected from the group consisting of HIV-1, HIV-2, SIV, FIV, EIAV and Visna. Lentiviruses are able to infect non-dividing (i.e. quiescent) cells which makes them attractive retroviral vectors for gene therapy. In a yet further embodiment, the retrovirus is HIV-1 or is derived from HIV-1. The genomic structure of some retroviruses may be found in the art. For example, details on HIV-1 may be found from the NCBI Genbank (Genome Accession No. AF033819). HIV-1 is one of the best understood retroviruses and is therefore often used as a retroviral vector.

Retroviral Sequences:

The nucleic acid sequences common to all retroviruses may be explained in more detail, as follows:

Long Terminal Repeats (LTRs): The basic structure of a retrovirus genome comprises a 5′-LTR and a 3′-LTR, between or within which are located the genes required for retroviral production. The LTRs are required for retroviral integration and transcription. They can also act as promoter sequences to control the expression of the retroviral genes (i.e. they are cis-acting sequences). The LTRs are composed of three sub-regions designated U3, R, U5: U3 is derived from the sequence unique to the 3′ end of the RNA; R is derived from a sequence repeated at both ends of the RNA; and U5 is derived from the sequence unique to the 5′ end of the RNA. Therefore, in one embodiment, the nucleic acid vector additionally comprises a 5′- and 3′-LTR. In a further embodiment, the U5 region of the 3′ LTR can be deleted and replaced with a non-HIV-1 polyA tail (see Hanawa et al. (2002) Mol. Ther. 5(3): 242-51).

In an embodiment of the invention the LTRs present in the nucleic acid vectors of the invention can be self inactivating LTRs (termed SIN LTRs).

In a further embodiment of the invention the nucleic acid modules of the invention can also comprise additional retrovirus nucleic acid sequences including those encoding gag, pol and env proteins (or functional substitute thereof), cis-acting sequences such as those encoding cPPT (central polypurine tract) and RRE (rev response element) as the presence of these will further increase virus titre.

In order to address safety concerns relating to the generation of replication-competent virus, a self-inactivating (SIN) vector has been developed by deleting a section in the U3 region of the 3′ LTR, which includes the TATA box and binding sites for transcription factors Sp1 and NF-κB (see Miyoshi et al. (1998) J. Virol. 72(10):8150-7). The deletion is transferred to the 5′ LTR after reverse transcription and integration in infected cells, which results in the transcriptional inactivation of the LTR. This is known as a self-inactivating lentiviral-based vector system which may be included in the present invention.

ψ: Encapsidation of the retroviral RNAs occurs by virtue of a ψ (psi) sequence located at the 5′ end of the retroviral genome. It is also well known in the art that sequences downstream of the psi sequence and extending into the gag coding region are involved in efficient retroviral vector production (see Cui et al. (1999) J. Virol. 73(7): 6171-6176). In one embodiment, the nucleic acid vector additionally comprises a ψ (psi) sequence.

Primer Binding Site (PBS): The retroviral genome contains a PBS which is present after the U5 region of the 5′-LTR. This site binds to the tRNA primer required for initiation of reverse transcription. In one embodiment, the nucleic acid vector additionally comprises a PBS sequence.

PPT: Retroviral genomes contain short stretches of purines, called polypurine tracts (PPTs), near the 3′ end of the retroviral genome. These PPTs function as RNA primers for plus-strand DNA synthesis during reverse transcription. Complex retroviruses (such as HIV-1) contain a second, more centrally located PPT (i.e. a central polypurine tract (cPPT)) that provides a second site for initiation of DNA synthesis. Retroviral vectors encoding a cPPT have been shown to have enhanced transduction and transgene expression (see Barry et al. (2001) Hum. Gene Ther. 12(9):1103-8). In one embodiment, the nucleic acid vector additionally comprises a 3′-PPT sequence and/or a cPPT sequence.

The genomic structure of the non-coding regions described above are well known to a person skilled in the art. For example, details on the genomic structure of the non-coding regions in HIV-1 may be found from the NCBI Genbank with Genome Accession No. AF033819, or for HIV-1 HXB2 (a commonly used HIV-1 reference strain) with Genome Accession No. K03455. In one embodiment, the non-coding regions are derived from the sequences available at Genome Accession No. K03455, for example from base pairs 454-1126 (for R-U5-PBS-Gag), 7622-8479 (for RRE) or 7769-8146 (for RRE), 4781-4898 (for cPPT), 9015-9120 & 9521-9719 (for dNEF-PPT-sinU3-R-U5).

Gag/pol: The expression of gag and pol genes relies on a translational frameshift between gag and gagpol. Both are polyproteins which are cleaved during maturation. The major structural matrix, capsid, and nucleocapsid proteins of the retroviral vector are encoded by gag. The pol gene codes for the retroviral enzymes: i) reverse transcriptase, essential for reverse transcription of the retroviral RNA genome to double stranded DNA, ii) integrase, which enables the integration of the retroviral DNA genome into a host cell chromosome, and iii) protease, that cleaves the synthesized polyprotein in order to produce the mature and functional proteins of the retrovirus. In one embodiment, the retroviral nucleic acid sequence encoding the gag and pot proteins is derived from the HIV-1 HXB2 sequence, which is available at Genome Accession No. K03455, for example from base pairs 790-5105.

Env: The env (“envelope”) gene codes for the surface and transmembrane components of the retroviral envelope (e.g. glycoproteins gp120 and gp41 of HIV-1) and is involved in retroviral-cell membrane fusion. In order to broaden the retroviral vector's tissue tropism, the retroviral vectors described herein may be pseudotyped with an envelope protein from another virus. Pseudotyping refers to the process whereby the host cell range of retroviral vectors, including lentiviral vectors, can be expanded or altered by changing the glycoproteins (GPs) on the retroviral vector particles (e.g. by using GPs obtained from or derived from other enveloped viruses or using synthetic/artificial GPs). The most commonly used glycoprotein for pseudotyping retroviral vectors is the Vesicular stomatitis virus GP (VSVg), due to its broad tropism and high vector particle stability. However, it will be understood by the skilled person that other glycoproteins may be used for pseudotyping (see Cronin et al. (2005) Curr. Gene Ther. 5(4):387-398, herein incorporated by reference in its entirety). The choice of virus used for pseudotyping may also depend on the type of cell and/or organ to be targeted because some pseudotypes have been shown to have tissue-type preferences.

In one embodiment of the invention, the env protein or a functional substitute thereof is obtained from or derived from a virus selected from a Vesiculovirus (e.g. Vesicular stomatitis virus), Lyssavirus (e.g. Rabies virus, Mokola virus), Arenavirus (e.g. Lymphocytic choriomeningitis virus (LCMV)), Alphavirus (e.g. Ross River virus (RRV), Sindbis virus, Semliki Forest virus (SFV), Venezuelan equine encephalitis virus), Filovirus (e.g. Ebola virus Reston, Ebola virus Zaire, Lassa virus), Alpharetrovirus (e.g. Avian leukosis virus (ALV)), Betaretrovirus (e.g. Jaagsiekte sheep retrovirus (JSRV)), Gammaretrovirus (e.g. Moloney Murine leukaemia virus (MLV), Gibbon ape leukaemia virus (GALV), Feline endogenous retrovirus (RD114)), Deltaretrovirus (e.g. Human T-lymphotrophic virus 1 (HTLV-1)), Spumavirus (e.g. Human foamy virus), Lentivirus (e.g. Maedi-visna virus (MVV)), Coronavirus (e.g. SARS-CoV), Respirovirus (e.g. Sendai virus, Respiratory syncytia virus (RSV)), Hepacivirus (e.g. Hepatitis C virus (HCV)), Influenzavirus (e.g. Influenza virus A) and Nucleopolyhedrovirus (e.g. Autographa californica multiple nucleopolyhedrovirus (AcMNPV)). In a further embodiment, the env protein or a functional substitute thereof is obtained from or derived from Vesicular stomatitis virus. In this embodiment, the Vesicular stomatitis virus glycoprotein (VSVg) protein may be used which enables the retroviral particles to infect a broader host cell range and eliminates the chances of recombination to produce wild-type envelope proteins. In a further embodiment, the retroviral nucleic acid sequence encoding the env protein or a functional substitute thereof, is derived from the sequence available at Genome Accession No. J02428.1, for example from base pairs 3071 to 4720.

The structural genes described herein are common to all retroviruses. Further auxiliary genes may be found in different types of retrovirus. For example, lentiviruses, such as HIV-1, contain six further auxiliary genes known as rev, vif, vpu, vpr, nef and tat. Other retroviruses may have auxiliary genes which are analogous to the genes described herein, however they may not have always been given the same name as in the literature. References such as Tomonaga and Mikami (1996) J. Gen. Virol. 77(Pt 8):1611-1621 describe various retrovirus auxiliary genes.

Rev: The auxiliary gene rev (“regulator of virion”) encodes an accessory protein which binds to the Rev Response element (RRE) and facilitates the export of retroviral transcripts. The gene's protein product allows fragments of retroviral mRNA that contain the Rev Responsive element (RRE) to be exported from the nucleus to the cytoplasm. The RRE sequence is predicted to form a complex folded structure. This particular role of rev reflects a tight coupling of the splicing and nuclear export steps. In one embodiment, nucleic acid vector comprises an RRE sequence. In a further embodiment, the RRE sequence is derived from HIV-1 HXB2 sequence, which is available at Genome Accession No. K03455, for example from base pairs 7622 to 8479, or 7769 to 8146, in particular base pairs 7622 to 8479.

Rev binds to RRE and facilitates the export of singly spliced (env, vif, vpr and vpu) or non-spliced (gag, pol and genomic RNA) viral transcripts, thus leading to downstream events like gene translation and packaging (see Suhasini and Reddy (2009) Curr. HIV Res. 7(1): 91-100). In one embodiment, the nucleic acid vector additionally comprises the auxiliary gene rev or an analogous gene thereto (i.e. from other retroviruses or a functionally analogous system). Inclusion of the rev gene ensures efficient export of RNA transcripts of the retroviral vector genome from the nucleus to the cytoplasm, especially if an RRE element is also included on the transcript to be transported. In a further embodiment, the rev gene comprises at least 60% sequence identity, such as at least 70% sequence identity to base pairs 970 to 1320 of Genome Accession No. M11840 (i.e. HIV-1 clone 12 cDNA, the HIVPCV12 locus). In an alternative embodiment, the rev gene comprises at least 60% sequence identity, such as at least 70%, 80%, 90% or 100% sequence identity to base pairs 5970 to 6040 and 8379 to 8653 of Genome Accession No. K03455.1 (i.e. Human immunodeficiency virus type 1, HXB2).

Auxiliary genes are thought to play a role in retroviral replication and pathogenesis, therefore many current viral vector production systems do not include some of these genes. The exception is rev which is usually present or a system analogous to the rev/RRE system is potentially used. Therefore, in one embodiment, the nucleic acid sequences encoding one or more of the auxiliary genes vpr, vif, vpu, tat and nef, or analogous auxiliary genes, are disrupted such that said auxiliary genes are removed from the RNA genome of the retroviral vector particle or are incapable of encoding functional auxiliary proteins. In a further embodiment, at least two or more, three or more, four or more, or all of the auxiliary genes vpr, vif, vpu, tat and nef, or analogous auxiliary genes, are disrupted such that said auxiliary genes are removed from the RNA genome of the retroviral vector particle or are incapable of encoding functional auxiliary proteins. Removal of the functional auxiliary gene may not require removal of the whole gene; removal of a part of the gene or disruption of the gene will be sufficient.

It will be understood that the nucleic acid sequences encoding the replication defective retroviral vector particle may be the same as, or derived from, the wild-type genes of the retrovirus upon which the retroviral vector particle is based, i.e. the sequences may be genetically or otherwise altered versions of sequences contained in the wild-type virus. Therefore, the retroviral genes incorporated into the nucleic acid vectors or host cell genomes, may also refer to codon-optimised versions of the wild-type genes.

Additional Components:

The nucleic acid modules of the invention may comprise further additional components. These additional features may be used, for example, to help stabilize transcripts for translation, increase the level of gene expression, and turn on/off gene transcription.

In particular, each of the nucleic acid sequences present in the modules of the invention e.g. retroviral nucleic acid sequences may be arranged as individual expression constructs within the nucleic acid module.

Gene Therapy:

The retroviral vector particles produced by introducing the modules of the invention or vectors of the invention (comprising the modules of the invention) into cultures of host cells (e.g. mammalian host cells) may be used in methods of gene therapy. Therefore, the nucleic acid module comprises one or more transgenes e.g. 1, 2 or 3 transgenes. Such transgenes may be a therapeutically active gene which encodes a gene product which may be used to treat or ameliorate a target disease. The transgene may encode, for example, an antisense RNA, a ribozyme, a protein (for example a tumour suppressor protein), a toxin, an antigen (which may be used to induce antibodies or helper T-cells or cytotoxic T-cells) or an antibody (such as a single chain antibody). In one embodiment, the transgene encodes beta globin, Cas-9, enzymes such as Arylsulphatase A, Galactocerebrosidase, Dyrstrophin. In another embodiment the transgene can encode T cell receptors (TCRs) or chimeric antigen receptors (CARs) such as those specific for tumour antigens e.g. NY-ESO-1.

Lentiviral Vectors:

Lentiviral vectors, such as those based upon Human Immunodeficiency Virus Type 1 (HIV-1) are widely used in gene therapy as they are able to integrate into non-proliferating cells. Viral vectors can be made replication defective by splitting the viral genome into separate parts, e.g., by placing on separate plasmids. For example, the so-called first generation of lentiviral vectors, developed by the Salk Institute for Biological Studies, was built as a three-plasmid expression system consisting of a packaging expression cassette, the envelope expression cassette and the vector expression cassette. The “packaging plasmid” contains the entire gag-pol sequences, the regulatory (tat and rev) and the accessory (vif, vpr, vpu, nef) sequences. The “envelope plasmid” holds the Vesicular stomatitis virus glycoprotein (VSVg) in substitution for the native HIV-1 envelope protein, under the control of a cytomegalovirus (CMV) promoter. The third plasmid (the “transfer plasmid”) carries the Long Terminal Repeats (LTRs), encapsidation sequence e.g. packaging sequence (ψ), the Rev Response Element (RRE) sequence and the CMV or RSV promoter to express the transgene inside the host cell.

The second lentiviral vector generation was characterized by the deletion of the virulence sequences vpr, vif, vpu and nef. The packaging vector was reduced to gag, pol, tat and rev genes, therefore increasing the safety of the system.

To improve the lentiviral system, the third-generation vectors have been designed by removing the tat gene from the packaging construct and inactivating the LTR from the vector cassette, therefore reducing problems related to insertional mutagenesis effects.

The various lentivirus generations are described in the following references: First generation: Naldini et al. (1996) Science 272(5259): 263-7; Second generation: Zufferey et al. (1997) Nat. Biotechnol. 15(9): 871-5; Third generation: Dull et al. (1998) J. Virol. 72(11): 8463-7, all of which are incorporated herein by reference in their entirety. A review on the development of lentiviral vectors can be found in Sakuma et al. (2012) Biochem. J. 443(3): 603-18 and Picanço-Castro et al. (2008) Exp. Opin. Therap. Patents 18(5):525-539.

The term “non-mammalian origin of replication” refers to a nucleic acid sequence where replication is initiated and which is derived from a non-mammalian source. This enables the nucleic acid vectors of the invention to stably replicate and segregate alongside endogenous chromosomes in a suitable host cell (e.g. a microbial cell, such as a bacterial or yeast cell) so that it is transmittable to host cell progeny, except when the host cell is a mammalian host cell. In mammalian host cells, nucleic acid vectors with non-mammalian origins of replication will either integrate into the endogenous chromosomes of the mammalian host cell or be lost upon mammalian host cell replication. For example, nucleic acid vectors with non-mammalian origins of replication such as bacterial artificial chromosomes (BAC), P1-derived artificial chromosome (PAC), cosmids or fosmids, are able to stably replicate and segregate alongside endogenous chromosomes in bacterial cells (such as E. coli), however if they are introduced into mammalian host cells, the BAC, PAC, cosmid or fosmid will either integrate or be lost upon mammalian host cell replication. Yeast artificial chromosomes (YAC) are able to stably replicate and segregate alongside endogenous chromosomes in yeast cells, however if they are introduced into mammalian host cells, the YAC will either integrate or be lost upon mammalian host cell replication.

Bacterial Artificial Chromosomes:

The term “bacterial artificial chromosome” or “BAC” refers to a DNA construct derived from bacterial plasmids which is able to hold a large insert of exogenous DNA. They can usually hold a maximum DNA insert of approximately 350 kb. BACs were developed from the well characterised bacterial functional fertility plasmid (F-plasmid) which contains partition genes that promote the even distribution of plasmids after bacterial cell division. This allows the BACs to be stably replicated and segregated alongside endogenous bacterial genomes (such as E. coli). The BAC usually contains at least one copy of an origin of replication (such as the oriS or oriV gene), the repE gene (for plasmid replication and regulation of copy number) and partitioning genes (such as sopA, sopB, parA, parB and/or parC) which ensures stable maintenance of the BAC in bacterial cells. BACs are naturally circular and supercoiled which makes them easier to recover than linear artificial chromosomes, such as YACs. They can also be introduced into bacterial host cells relatively easily, using simple methods such as electroporation.

In one embodiment, the bacterial artificial chromosome comprises an oriS gene. In one embodiment, the bacterial artificial chromosome comprises a repE gene. In one embodiment, the bacterial artificial chromosome comprises partitioning genes. In a further embodiment, the partitioning genes are selected from sopA, sopB, parA, parB and/or parC. In a yet further embodiment, the bacterial artificial chromosome comprises a sopA and sopB gene.

BACs for use according to the invention and that can be further modified as described in the present invention may be obtained from commercial sources, for example the pSMART BAC from LUCIGEN™ (see Genome Accession No. EU101022.1 for the full back bone sequence). This BAC contains the L-arabinose “copy-up” system which also contains the oriV medium-copy origin of replication, which is active only in the presence of the TrfA replication protein. The gene for TrfA may be incorporated into the genome of bacterial host cells under control of the L-arabinose inducible promoter araC-P_{BAD} (see Wild et al. (2002) Genome Res. 12(9): 1434-1444). Addition of L-arabinose induces expression of TrfA, which activates oriV, causing the plasmid to replicate to up to 50 copies per cell.

Therefore, in this context, the nucleic acid vectors comprising a nucleic acid module of the invention such as the modified BACs or transfer vectors, of the invention act as reservoirs of DNA (i.e. for the genes essential for retroviral production) which can be easily transferred into mammalian cells to generate stable cell lines suitable for retroviral production. Examples of non-mammalian origins of replication include bacterial origins of replications, such as oriC, oriV or oriS, or yeast origins of replication, also known as Autonomously Replicating Sequences (ARS elements).

The nucleic acid vectors (e.g. BACs) comprising a nucleic acid module of the present invention comprise a non-mammalian origin of replication and are for example able to hold at least 25 kilobases (kb) of DNA. In one embodiment, the nucleic acid vector has the ability to hold at least 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340 or 350 kb of DNA. It will be understood that references to “ability to hold” has its usual meaning and implies that the upper limit for the size of insert for the nucleic acid vector is not less than the claimed size (i.e. not less than 25 kb of DNA).

In an embodiment of the invention such nucleic acid vectors are also not bacteriophages which generally only hold maximum inserts of 5-11 kb. Therefore, in one embodiment the nucleic acid vector comprising a module of the invention is not a natural plasmid, bacteriophage or episome.

The term “endogenous chromosomes” refers to genomic chromosomes found in the host cell prior to generation or introduction of an exogenous nucleic acid vector, such as a bacterial artificial chromosome. The terms “transfection”, “transformation” and “transduction” as used herein, may be used to describe the insertion of the non-mammalian or viral vector into a target cell. Insertion of a vector is usually called transformation for bacterial cells and transfection for eukaryotic cells, although insertion of a viral vector may also be called transduction. The skilled person will be aware of the different non-viral transfection methods commonly used, which include, but are not limited to, the use of physical methods (e.g. electroporation, cell squeezing, sonoporation, optical transfection, protoplast fusion, impalefection, magnetofection, gene gun or particle bombardment), chemical reagents (e.g. calcium phosphate, highly branched organic compounds or cationic polymers) or cationic lipids (e.g. lipofection). Many transfection methods require the contact of solutions of plasmid DNA to the cells, which are then grown.

The term “promoter” refers to a sequence that drives gene expression. In order to drive a high level of expression, it may be beneficial to use a high efficiency promoter, such as a non-retroviral, high efficiency promoter.

The term “polyA signal” refers to a polyadenylation signal sequence, for example placed 3′ of a transgene, which enables host factors to add a polyadenosine (polyA) tail to the end of the nascent mRNA during transcription. The polyA tail is a stretch of up to 300 adenosine ribonucleotides which protects mRNA from enzymatic degradation and also aids in translation. Accordingly, the nucleic acid modules of the present invention (or vectors comprising these modules) may include a polyA signal sequence such as the human beta globin or rabbit beta globin polyA signals, the simian virus 40 (SV40) early or late polyA signals, the human insulin polyA signal, or the bovine growth hormone polyA signal. In one embodiment, the polyA signal sequence is the human beta globin polyA signal.

The term “intron sequence” refers to a nucleotide sequence which is removed from the final gene product by RNA splicing. The use of an intron downstream of the enhancer/promoter region and upstream of the cDNA insert has been shown to increase the level of gene expression. The increase in expression depends on the particular cDNA insert. Accordingly, the nucleic acid modules of the present invention (or vectors comprising these modules) may include introns such as human beta globin intron, rabbit beta globin intron II or a chimeric human beta globin-immunoglobulin intron. In one embodiment, the intron is a human beta globin intron and/or a rabbit beta globin intron II.

The term “packaging cell line” refers to a cell line which is capable of expressing gag and pot protein and envelope glycoprotein genes. Alternatively, the term “producer cell line” refers to a packaging cell line which is also capable of expressing a transfer vector containing a transgene of interest.

The term “transiently transfected” refers to transfected cells where the target nucleic acids (i.e. retroviral genes) are not permanently incorporated into the cellular genome. Therefore, the effects of the nucleic acids within the cell last only a short amount of time.

The term “stably transfected” refers to transfected cells where the target nucleic acids (i.e. retroviral genes) are permanently incorporated into the cellular genome. Therefore, the effects of the nucleic acids within the cell last only a short amount of time.

Splice Donor Sequence:

All retroviruses contain a major splice donor in the 5′ UTR (untranslated region) which enables the generation of multiple spliced isoforms encoding different retroviral gene products where unspliced mRNA serves as both gag mRNA and genomic RNA. This is conserved feature of all retroviruses and is well known and described in the art, (e.g. Mueller, Klaver, Berkhout, & Das, 2015; J. of General Virology, vol 96, Issue 11, pp 3389-3395).

Splice Acceptor Sequence:

The splice acceptor consensus sequence is a pyrimidine rich run of nucleotides followed by an AG (the acceptor nucleotides) and typically followed by a G or A (Brent & Guigó, Current Opinion in Structural Biology, 14, pp 264-272, 2004). Such sequences are well known and described in the art, e.g. (Martin Stoltzfus, 2009, Chapter 1 Regulation of HIV-1 Alternative RNA Splicing and Its Role in Virus Replication. In Adv Virus Res (Vol. Volume 74, pp. 1-40): Academic Press). Since these sequence requirements are not stringent they often occur in sequences. Where these sequences appear downstream of the major splice donor in vector genomes they have the potential to act as splice acceptors.

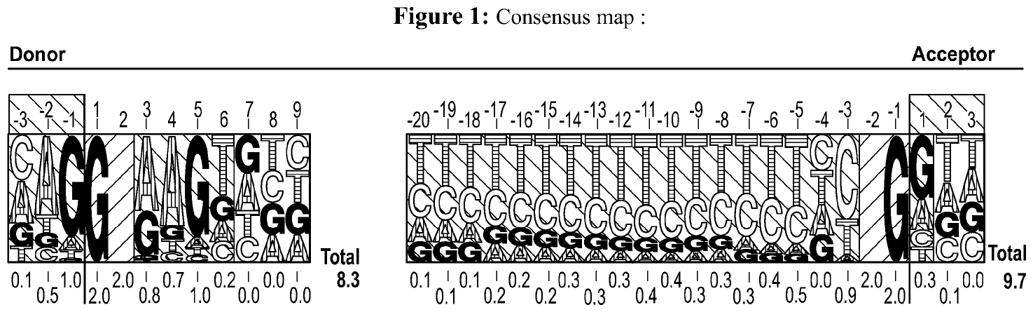

FIG. 1: shows a Consensus map for splice acceptors (Brent & Guigó, 2004; Recent advances in gene structure prediction. Current Opinion in Structural Biology, 14(3), 264-272). The different sized letters in FIG. 1 show how conserved that particular base is across all known splice sites. For the splice donor there must be a GT, for the acceptor there must be an AG. As is well known in RNA T will be replaced by U. The sequence around these is less stringent. A better match to consensus would mean e.g. AGGTAAG for the splice donor, ATATATATATATCAGG for the acceptor for example. In addition to these sequence requirements, the potential splice sites must have a secondary structure that makes it accessible to the splicing machinery and is difficult to predict in silica. Splice site prediction algorithms scan sequences looking for the primary sequence requirements and assign a score based on how well the actual sequence matches the consensus. For MaxEntScan the scores range from 1 (weakest match) to 13 (strongest match). Splice site prediction algorithms that can be used are described herein.

Nucleic Acid Vectors:

The vectors (such as plasmids or BACs) of the invention which comprise a nucleic acid module of the invention with an attenuated splice acceptor site within the promoter driving transgene expression, thereby reducing or eliminating production of spliced vector transcripts competent for transgene expression is advantageous as it reduces or eliminates expression of the transgene in producer cells thereby reducing possible undesirable effects of the transgene in the producer cells such as cytotoxic effects.

The use of vectors e.g. plasmids or BACs of the invention comprising a nucleic acid module of the invention which has a modified promoter with an attenuated splice acceptor site as described herein (e.g. attenuated by modification or full or partial deletion) therefore provides advantages in the generation of retroviral packaging and producer cell lines and also transient cell lines. Such suitable promoters with an attenuated splice acceptor site as described herein can therefore be those promoters which comprise a splice acceptor site that is active in the context of retroviral vector transcripts such that it accepts splicing from transcripts originating at the 5′LTR to create a spliced product that is competent for transgene expression. Examples include a promoter which is selected from SV-40, PGK, EFS, SFFV, and the Ubc promoter. The promoters can be human promoters or they can be orthologues from other species. In an embodiment said promoter is the PGK promoter for example the human PGK promoter. The work described herein has shown that the human PGK promoter comprises an active splice acceptor at it's 3′ terminus and here the active splice acceptor site identified can be fully or partially deleted. In this example where the promoter is the PGK promoter we have found that the last 2 base pairs which comprise the active splice acceptor are adenine and guanine (AG) and these 2 base pairs i.e. AG can both be deleted or alternatively there can be a partial deletion such that only either A or G is deleted or substituted or disrupted by insertional mutagenesis. When the A or G is substituted suitable substitutions can be any of the other nucleotides making up DNA for example in place of G either A, thymine (T) or cytosine (C) can be utilised. Other mutations within the 23 bp splice acceptor (where AG is positions 19 and 20) which reduce splice acceptor activity are also possible.

In an embodiment the modified PGK promoter can have the nucleic acid sequence shown in SEQ ID NO 19. In this sequence the guanine which comprises the active splice acceptor site as described above is deleted (this is the PGKnoSA2).

In an embodiment the modified PGK promoter can have the nucleic acid sequence shown in SEQ ID NO 20. In this sequence the both the adenine and guanine which comprises the active splice acceptor site as described above is deleted (this is the PGKnoSA).

Examples of other suitable promoters for use according to the invention which comprise cryptic splice acceptors (active in a lentivirus/retrovirus context) suitable for full or partial deletion according to the invention include the SV40 promoter e.g. the SV40 229 promoter which has previously been shown to contain a cryptic splice acceptor site this is activated in a retroviral context (this is described in Anson and Fuller, 2003, Rational development of a HIV-1 gene therapy vector. J Gene, 5, (10) pp 829-838).

Other promoters that can also be used (and modified) according to the invention include EFS (EF1α short), Ubc promoter, SFFV promoter (see Table 1).

In an embodiment the inventions also provides any of the modified promoters as described herein (e.g. for use in a module or vector encoding a transgene). For example the invention provides promoters with the nucleic acid sequence shown in: SEQ ID NO 19 and SEQ ID NO 20,

In one embodiment of the invention the promoter used is a wild type promoter or natural promoter that is then modified as described, e.g. by mutagenesis techniques. Alternatively the promoter may be based on a wild type sequence which is then modified as described herein but the mutagenesis is performed in-silico and the final modified nucleic acid sequence is synthesized using techniques well known in the art for such synthesis.

The modification of the promoter refers to modification from the wild type promoter and this can include in silico synthesis of the promoter as well as physical mutagenesis of the promoter.

Identification of splice acceptors may be achieved through sequencing mRNA of cells transfected with a lentivirus transfer plasmid (or similar design) containing the promoter of interest. For example, to experimentally determine whether there is an active splice acceptor in the EFS promoter (Schambach et al., 2006, Mol. Therapy, vol 13, no 2 pp 391-400) when in the context of a lentivirus transfer plasmid, one could transfect a lentivirus vector transfer plasmid with a transgene controlled by the EFS promoter. The mRNA from these transfected cells could then be sequenced by various techniques. This could include targeted approaches such as sequencing of PCR products from 5′RACE or amplification of junctions following RT-PCR, or non-targeted approaches such as RNAseq. Alignment to the transfer plasmid will show exon sequences and allow identification of any active splice acceptors at the 3′ junctions of introns.

Alternatively, splice acceptors may be predicted in silico.

MaxEntScan:

Multiple splice site prediction algorithms are available to facilitate this, for example Position Weight Matrix (PWM) based analysis, Maximal Dependence Decomposition (MDD), Markov Models or Maximum Entropy Distributions (MED) To date, the most unbiased approach was shown to be MED. This is a method that only uses features from available data (spliced RNA in human cells) considering bases in the positions adjacent to the splice sites (see In silico tools for splicing defect prediction—A survey from the viewpoint of end-users, Xueqiu Jian, Eric Boerwinkle and Xiaoming Liu. Genet Med. 2014.) This MED approach is implemented in the MaxEntScan—which is a “maximum entropy scanning tool” as described in Yeo & Burge, J. Comput Biol. 2004; 11 (2-3) pp 377-94) which analyses 9 bases around the 5′ splice donor sites and 23 bases around the splice acceptor sites. Using MED, it produces a maximum entropy score as log-odds ratio for each identified site, where a higher score implies a higher probability of the sequence being a true splice site.

Predicted splice acceptors within a promoter can be identified. Those that would cause the first start codon (ATG) in the spliced mRNA to belong to (or be in frame with) the transgene could then be mutated to abolish or weaken the splice acceptor. However, each mutation has the potential to alter promoter activity and must therefore be tested empirically.

TABLE 1
Example splice site prediction
for commonly used promoters

| Promoter | | | Transgene |
|---|---|---|---|
| and | | | would |
| splice | | | become |
| acceptor | | Splice | first |
| location | | acceptor | open |
| (from 5′ | | score | reading |
| boundary) | Motif | (MaxEntScan) | frame? |
| PGK-492 | TCACCGACCTC | 8.69 | Yes- |
| | TCTCCCCAG | (dependent | confirmed |
| | | on 3 ntds | experimen- |
| | | after | tally |
| | | AG) | |
| EFS(EF1a | CGCAACGGGTT | 4.54 | Yes |
| short)-184 | TGCCGCCAGAAC | | |
| EF1a | gtttttttcttc | 13 | Yes |
| | catttcAGgtg | | |
| Ube-902 | TAGGCTTTTCTC | 7.48 | No |
| | CGTCGCAGGAC | | |
| Ube-1134 | GGTCAATATGT | 4.06 | Yes |
| | AATTTTCAGT | | |
| | GT | | |
| Ube-1189 | GTTTTTGGCTTT | 5.18 | Yes |
| | TTTGTTAGAC | (dependent | |
| | | on 3 | |
| | | ntds after | |
| | | AG) | |
| SFFV-371 | CCCCTCACTCGG | 3.01 | Yes |
| | CGCGCCAGTCC | | |
| SFFV-374 | GCGCGCCAGTCC | 1.77 | Yes |
| | TCCGACAGACT | | |
| SV40-171 | CCGCCCCTAACT | 3.98 | No |
| | CCGCCCAGTTC | | |
| SV40-229 | AATTTTTTTTAT | 6.32 | Yes |
| | TTATGCAGAGG | | |
| CMV-252 | GGGACTTTCCTA | 2.94 | No |
| | CTTGGCAGTAC | | |

In the above table, motifs identified in silica using MaxEntScan are shown. Where an AG was present in the final 3 nucleotides (ntds) of a promoter region, additional bases were added since MaxEntScan requires the AG to be in positions 19 and 20 of a 23-mer to be scored (applicable to PGK-492 where cct was added, and Ubc-1189 where a G was added). Using SV40 as an example, 2 potential splice acceptors are shown at positions 171 and 229. The motif at position 171 would not lead to transgene expression since there is an ATG (start codon) downstream of that has a stop codon (TAA) in frame just 3 codons downstream of it (AGTTCCGCCCATTCTCCGCCCCATGGCTGACTAA). However, the motif at position 229 does not have an ATG downstream within the promoter sequence, meaning the 5′ ATG (start codon) from a transcript that used this splice acceptor would belong to the transgene (and therefore such a transcript would be competent for transgene protein expression).

Alternatively the promoter comprising an active splice acceptor can be modified: Modifications which can be used according to the invention to reduce splicing can be for example modification of the consensus sequence around active splice acceptor site such that its sequence is weakened and thus has reduced tendency to be used as a splice acceptor. For example where the PGK promoter is used the consensus sequence around the active splice acceptor in the last 2 base pairs (AG base pairs) can be modified such that its weakened. The modifications in the consensus sequence (where the 3 bp downstream of the AG dinucleotide are cct) can be for example from TCACCGACCTCTCTCCCCAG(cct) to TCACCGACCTCTCCCCgAG(cct) as this weakens its MaxEntScore from 8.69 to <3 (below threshold to be scored as a potential splice acceptor).

Use of Splice Traps:

However, a possible disadvantage of the attenuation (e.g. by modification or deletion) of the active splice acceptor as described above is that such mutagenesis of sequences within a promoter sequence may also alter transgene expression in transduced cells, and this would need to be assessed for each promoter to determine whether the effect on transgene expression was significant.

Therefore, an alternative strategy to reduce transgene expression in producer cells (but maintain transgene expression in transduced cells) and that overcomes these limitations described above was devised and this involves the use of “splice traps” since the majority of transgene expression in producer cells has been shown to be due to spliced transcripts originating from the LTR.

A “splice trap” can be used in a nucleic acid module according to the invention and can be positioned upstream (i.e. 5′ of the transgene) or downstream (i.e. 3′ of the transgene), or between the major splice donor and transgene cassettes transcriptional start site or it can be positioned between the major splice donor and the site of polyadenylation.

Splice traps as described and defined herein are short nucleic acid sequences that include a splice acceptor site (e.g. a strong splice acceptor site) optionally followed by a minimal open reading frame (ORF) e.g. less than about 50 codons e.g. about 40 codons or about 30 codons, or about 20 or about 10 codons. Splice acceptor sites may be synthetic based on consensus sequences or may be naturally occurring known splice acceptors such as the splice acceptor from intron 1 of the human EF1a promoter (Kim, Uetsuki, Kaziro, Yamaguchi, & Sugano, Use of the human elongation factor 1 alpha promoter as a versatile and efficient expression system. Gene 1990, 91(2), 217-223).

In an embodiment of the invention such splice traps have a MaxEntScan score (determined as described in Yeo & Burge, J. Comput Biol. 2004; 11 (2-3) pp 377-94) greater than that of the splice acceptor in the promoter that leads to transgene expression in order to work optimally. Alternatively, the MaxEntScan splice acceptor score can be greater than about 8.7 e.g. greater than about 9, or 10, or 11 or 12 or 13.

In an embodiment of the invention a suitable splice trap has a nucleic acid sequence which is C and T rich at the 5′ end e.g. a sequence of greater than about 14, or greater than about 16 or greater than about 18 C and T nucleotides and has AG nucleotides at the 3′ end and optionally also has a MaxEnt Scan score be greater than about 8.7 e.g. greater than about 9, or 10, or 11 or 12 or 13.

Splice traps can be utilised for example upstream of any promoter for transgene expression. For example it is desirable to use such splice traps in the nucleic acid modules of the invention when promoters of said modules (i) are not screened for cryptic splice acceptor sites, or (ii) said promoters have been screened and are then found to contain a potential splice acceptor that's use would generate transcripts competent for transgene expression and that is located within a region which is expected to or does significantly contribute to promoter/transcriptional activity and therefore it may be undesirable to mutate the sequence.

Methods for assessing transgene expression are well known in the art and include for example qPCR to quantify specific transcripts isoforms, transcriptional reporter assays and transgene activity assays or detection using antibodies against the transgene in techniques such as western blot, ELISA, flow cytometry, immunofluorescence.

If a cryptic splice acceptor motif within a promoter is mutated and transgene expression is significantly altered according to an appropriate statistical test (e.g. T-test) such that expression levels in transduced cells have worsened efficacy then it can be desirable to use a splice trap instead of modification or deletion of the active splice acceptor.

Hence in one aspect of the invention there is provided a nucleic acid module that comprises at least one “splice trap” e.g. 2 or more splice traps as an alternative (or in addition) to the modified or deleted active splice acceptor.

In a further aspect of the invention there is provided a vector comprising a nucleic acid module that comprises at least one “splice trap” as described above.

The splice trap sequence can for example be positioned immediately upstream of the transgene cassette such that they are transcribed before the transgene cassette in LTR-derived mRNAs but are excluded from transgene cassette derived mRNA. Alternative locations between the major splice donor and transgene cassettes transcriptional start site may also be suitable. Alternatively, the splice trap sequence can be positioned anywhere between the major splice donor and polyA signal.

These sequences were designed to divert LTR-derived mRNAs that were destined to be spliced away from the transgene cassette and onto the splice trap. When used as a splice acceptor in conjunction with the major splice donor, the spliced mRNA would have a 5′ start codon belonging to the minimal ORF (or substitute) rather than the transgene. Minimal ORFs consist of a start codon followed by a stop codon. Since mammalian ribosomes utilise a 5′ ORF scanning mechanism for translational initiation and disassemble upon reaching a stop codon, downstream ORFs (e.g. transgene) are unlikely to be translated efficiently. Nevertheless, a second minimal ORF was positioned adjacent to the first, such that the ribosome must read through at least two start and stop codons prior to reaching transgene in any mRNA that has utilised the splice trap. Furthermore, to promote ribosomal assembly at the first minimal ORF a Kozak sequence was included. Hence, efficient translation of transgene from such a processed mRNA is unlikely. Alternatively, an ORF that doesn't encode for protein with known or expected function can be used instead of a minimal ORF. Sequences downstream of the splice trap (typically in the promoter) will often contain such a short ORF, in which case insertion of an additional ORF may not be necessary for transgene suppression. It may still be desirable to negate the potential for the expression of an unknown protein to impact the producer cell or vector characteristics.

Examples of splice traps that can be used in nucleic acid modules of the invention are detailed in the table below:

TABLE 2
Examples of Splice trap sequences tested

| | Sequence (splice | Origin of |
|---|---|---|
| | acceptor motif- | splice |
| Splice | Kozak sequence-2x | acceptor |
| trap | [Start-stop]) | motif |
| STI | TCCTTAACgggttttt | Splice acceptor |
| | tttttttttttcCAGg | consensus |
| | ttgccgccaccATGTA | |
| | AATGTAG | |
| ST2 | TCCTTAACggcccttc | Splice acceptor |
| | tccctttcttcCAGgt | consensus |
| | tgccgccaccATGTAA | alternative |
| | ATGTAG | sequence |
| ST3 | TTCGTTGACCGAATC | Human PGK promoter |
| | ACCGACCTCTCTCCC | |
| | CAGcctgccgccacc | |
| | ATGTAAATGTAG | |
| ST4 | Tcagtggttcaaagt | EF1a promoter |
| | ttttttcttccattt | |
| | cAGggccgccaccAT | |
| | GTAAATGTAG | |
| ST5 | accatgttcatgcct | CAG promoter |
| | tcttctttttcctac | |
| | AGctgccgccaccAT | |
| | GTAAATGTAG | |
| ST6 | aagttaagtaatagtc | SAO |
| | cctctctccaagctc | (Hildinger, Abel, |
| | acttacAGgccgccac | Ostertag, & Baum, |
| | cATGTAAATGTAG | 1999) |

ST6 sequence is described in Hildinger et al 1999; Design of 5′ untranslated sequences in retroviral vectors developed for medical use. Journal of Virology, 73(5), 4083-4089.

In an embodiment of the invention the splice traps utilised are selected from: ST1, ST2, ST4 and ST5 or functional fragments thereof. Such functional fragments (or variants) can comprise the underlined portions of the sequences ST1, ST2, ST4 and ST5 as detailed in Table 2 as these comprise: a CT rich region at the 5′ and AG at the 3′end. Any T can be replaced by a C and vice versa. Alternatively functional fragments (or variants) of ST1, ST2, ST4 and ST5 are which have a MaxEntScan splice acceptor score which is greater than about 8.7 e.g. greater than about 9, or 10, or 11 or 12 or 13.

TABLE 3
Characteristics of a splice trap

| | | Splice trap |
|---|---|---|
| | MaxEntScan | activity upstream of |
| Splice trap | acceptor score | PGK-GFP-WPRE |
| St1 | 12.59 | High |
| St2 | 12.54 | High |
| St3 | 8.69 | Low |
| St4 | 12.38 | High |
| St5 | 12.39 | High |
| ST6 | 3.16 | Low |

Also provided by the invention are methods of making nucleic acid vectors comprising the nucleic acid modules of the invention which methods comprise obtaining a nucleic acid vector e.g. a plasmid, retroviral transfer vector or a BAC (such as those described in WO 2017/089307), comprising the following retroviral nucleic acid sequences:

cis-acting viral elements such as LTRs, a packaging signal, a promoter for transgene expression operably linked said transgene, and also optionally retroviral nucleic acid sequences encoding gag, pol and env proteins (or functional substitute thereof) and also a non-mammalian origin of replication and then performing the step of (i) modifying the promoter for transgene expression by deleting (wholly or partially) the splice acceptor site or modifying it e.g. by insertion, deletion or substitution mutations such that it has reduced or eliminated function as a splice acceptor, thereby reducing or eliminating splicing at this location; and or (ii) inserting into said vector a “splice trap” such that it is positioned upstream and/or downstream of the transgene or is inserted between the major splice donor and transgene cassettes transcriptional start site or positioned between the major splice donor and the site of polyadenylation.

According to a further aspect of the invention, there is provided replication defective retroviral vector particles that are obtainable or obtained by the methods of the invention as defined herein.

According to a further aspect of the invention, there is provided the nucleic acid vectors comprising the modules as defined herein for use in a method of producing replication defective retroviral vector particles.

Use of Large Nucleic Acid Vectors:

The nucleic acid vectors (such as the artificial chromosomes e.g. BACs) comprising the nucleic acid modules of the present invention can be based on for example those that are described in WO 2017/089307 or those described in WO 2017/089308 wherein the nucleic acid vector e.g. BAC, comprises a non-mammalian origin of replication and the ability to hold at least 25 kilobases (kb) of DNA, characterized in that said nucleic acid vector comprises retroviral nucleic acid sequences encoding:

gag and pot proteins, and an env protein or a functional substitute thereof, and that further comprise a modified or fully or partially deleted splice acceptor site and/or a splice trap as described herein.

The vectors of WO 2017/089307 may also as detailed therein further comprise the RNA genome of a retroviral vector particle and/or the auxiliary gene rev or an analogous gene thereto or a functionally analogous system and/or a transcription regulation element.

In one embodiment the nucleic acid vectors comprising a nucleic acid module of the present invention may therefore be large nucleic acid vectors for example selected from bacterial artificial chromosome (BAC), a yeast artificial chromosome, a P1-derived artificial chromosome, fosmid or a cosmid.

The advantage of including all of the retroviral genes on such a large nucleic acid vector e.g. a BAC is that they can be prepared in microbial host cells (such as bacterial or yeast host cells) first, which are much easier to handle and manipulate, before being introduced into mammalian cells in a single step.

Host Cells:

According to yet a further aspect of the invention, there is provided a cell line comprising the nucleic acid vectors which comprise the nucleic acid modules of the present invention (such as the modified transfer vectors or modified BACs) as described herein which are integrated into a culture of mammalian host cell following transduction or transfection of the host cell line.

In one embodiment, the host cell is a mammalian cell. In a further embodiment, the mammalian cell is selected from a HEK 293 cell, HEK 6E cell, CHO cell, Jurkat cell, KS62 cell, PerC6 cell, HeLa cell, HOS cell, H9 cell or a derivative or functional equivalent thereof. In a yet further embodiment, the mammalian host cell is a HEK 293 cell, or derived from a HEK 293 cell. Such cells could be adherent cell lines (i.e. they grow in a single layer attached to a surface) or suspension adapted/non-adherent cell lines (i.e. they grow in suspension in a culture medium). In a yet further embodiment, the HEK 293 cell is a HEK 293T cell or a HEK 6E cell. The term “HEK 293 cell” refers to the Human Embryonic Kidney 293 cell line which is commonly used in biotechnology. In particular, HEK 293T cells are commonly used for the production of various retroviral vectors. Other examples of suitable commercially available cell lines include T REX™ (Life Technologies) cell lines.

The host cells transduced using the methods defined herein may be used to produce a high titre of retroviral vector.

References herein to the term “high titre” refer to an effective amount of retroviral vector or module/particle which is capable of transducing a target cell, such as a patient cell. In one embodiment, a high titre is in excess of 10⁶ TU/ml without concentration (TU=transducing units).

The skilled person will be aware that introducing the nucleic acid vector comprising the modules of the invention as described herein into the host cell may be performed using suitable methods known in the art, for example, lipid-mediated transfection (lipofection), microinjection, cell (such as microcell) fusion, electroporation, chemical-based transfection methods or microprojectile bombardment. It will be understood that the choice of method to use for introducing the nucleic acid vector can be chosen depending upon the type of mammalian host cell used. In one embodiment, introduction step (a) is performed using lipofection, electroporation or a chemical-based transfection method. In a further embodiment, the nucleic acid vector is introduced into the host cell by lipofection. In an alternative embodiment, the nucleic acid vector is introduced into the host cell by a chemical-based transfection method, such as calcium phosphate treatment. Calcium phosphate treatments are commercially available, for example from Promega.

It will be understood by the skilled person that the conditions used in the method described herein will be dependent upon the host cell used. Typical conditions, for example the culture medium or temperature to be used, are well known in the art (e.g. see Kutner et al. (2009) Nature Protocols 4(4); 495-505). In one embodiment, culturing step (b) is performed by incubating the mammalian host cell under humidified conditions. In a further embodiment, the humidified conditions comprise incubating the transfected cells at 37° C. at 5% CO₂. In one embodiment, culturing step (b) is performed using a culture medium selected from: Dulbecco's modified Eagle's medium (DMEM) containing 10% (vol/vol) fetal bovine serum (FBS) or serum-free UltraCULTURE™ medium (Lonza, Cat. No. 12-725F) or FreeStyle™ Expression medium (Thermo fisher, Cat. No. 12338 018).

In one embodiment, the method additionally comprises isolating the replication defective retroviral vector particle. For example, in one embodiment the isolating is performed by using a filter. In a further embodiment, the filter is a low-protein binding membrane (e.g. a 0.22 μm low-protein binding membrane or a 0.45 μm low-protein binding membrane), such as polyvinylidene fluoride (PVDF) or polyethersulfone (PES) artificial membranes.

In one embodiment, the replication defective retroviral vector particles are isolated no longer than 72 hours after introduction step (a). In a further embodiment, the replication defective retroviral vector particles are isolated between 48 and 72 hours after introduction step (a), for example at 48, 49, 50, 51, 52, 53, 54, 55, 56, 57, 58, 59, 60, 61, 62, 63, 64, 65, 66, 67, 68, 69, 70, 71 or 72 hours.

Once isolated, the retroviral vector particles may be concentrated for in vivo applications. Concentration methods include, for example, ultracentrifugation, precipitation or anion exchange chromatography. Ultracentrifugation is useful as a rapid method for retroviral vector concentration at a small scale. Alternatively, anion exchange chromatography (for example using Mustang Q anion exchange membrane cartridges) or precipitation (for example using PEG 6000) are particularly useful for processing large volumes of lentiviral vector supernatants.

According to a further aspect of the invention, there is provided a method of producing a replication defective retroviral vector particle, comprising:
(a) introducing a nucleic acid vector (e.g. a BAC as described herein) which comprises a nucleic acid module of the invention into a culture of mammalian host cells; and
(b) culturing the mammalian host cells under conditions in which the replication defective retroviral vector particle is produced,
(c) isolating said replication defective retroviral vector particles.

Such replication defective retroviral vector particles can then be formulated e.g. with suitable excipients such as cell culture medium for ex vivo use or suitable pharmaceutical excipients for use in therapy e.g. for administration to a subject such as a human subject in need thereof.

According to a further aspect of the invention, there is provided a replication defective retroviral vector particle that is obtainable or obtained by the methods of the invention as defined herein.

According to a further aspect of the invention, there is thus provided the nucleic acid vectors comprising a nucleic acid module of the invention as defined herein for use in a method of producing replication defective retroviral vector particles.

In one embodiment of the invention, multiple copies of the RNA genome of the retroviral vector particle are included in the nucleic acid vector. Multiple copies of the are expected to result in higher viral vector titre. For example, the nucleic acid vector may include two or more, such as three, four, five, six, seven, eight, nine or ten or more copies of the RNA genome of the retroviral vector particle.

The invention will now be described in further detail with reference to the following, non-limiting Examples.

### EXAMPLES

### Example 1: Deletion of the PGK Promoter Cryptic Splice Acceptor Site to Create PGKnoSA

Following identification of a cryptic splice acceptor which is found within the human PGK promoter, the AG dinucleotide (essential for splice acceptor activity) located at the 3′ terminus of the PGK promoter was targeted for deletion as is described below:

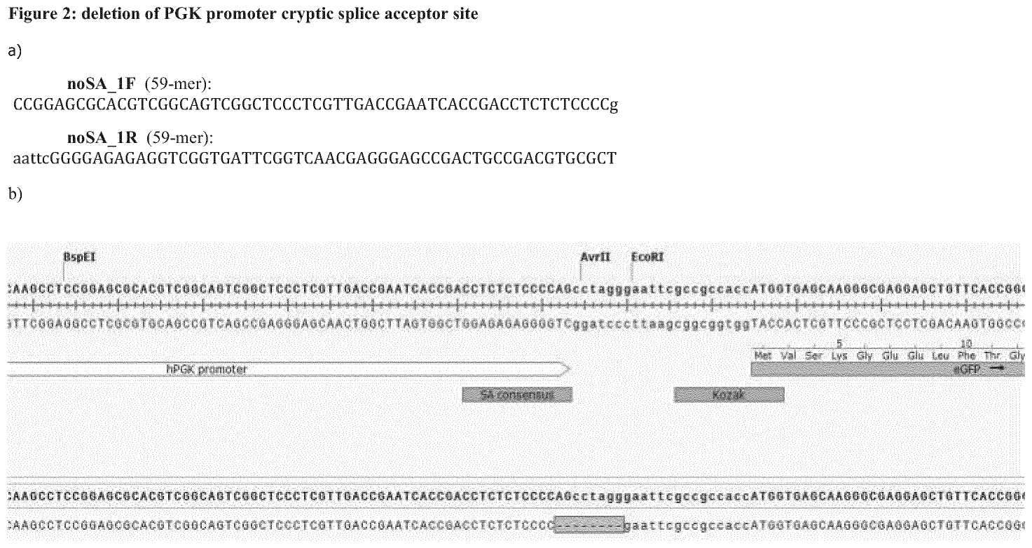

To achieve this deletion, oligonucleotides noSA_1F and noSA_1R were annealed together such that the annealed product had ‘sticky’ ends compatible with BspE1 and EcoRI restriction enzyme digested DNA (see FIG. 2a).

The annealed product was ligated into a 3^{rd} generation self-inactivating lentivirus vector plasmid, BmodT-PGW, which had been digested with BspEI and EcoRI restriction enzymes. The resulting plasmid, termed BmodT-PGKnoSA GW, had 8 base pairs deleted (as is shown in FIG. 2b): the AG dinucleotide of the cryptic splice acceptor and the adjacent AvrII restriction site.

The AvrII site, located immediately 3′ of the PGK promoter, was removed since it contains an AG dinucleotide that could have effectively replaced the deleted dinucleotide from PGK and, therefore, splice accepting activity at this site may have continued. A sequence alignment between BmodT-PGW and BmodT-_PGKnoSA GW is shown in FIG. 2b. The mutation was confirmed by restriction digest and Sanger sequencing. Aside from the targeted 8 bp deletion, the constructs are identical.

### Example 2 Determining the Impact of Deleting the PGK Promoter Cryptic Splice Acceptor on Transgene Expression and Titre

To evaluate the impact of PGKnoSA (SEQ ID NO 20) on transgene expression, each transfer plasmid (BmodT-PGW and BmodT-PGKnoSA GW) was co-transfected with BAC1-pax, a construct which encodes all 3^{rd} generation packaging components (Rev, Gag, pol and VSVg) under the control of the Tet repressor (Tet-On configuration), (as described in WO 2017/089307 and in WO 2017/089308).

Transfer plasmid was mixed with BAC1-pax in equimolar amounts and complexed with PEIpro. The DNA/PEI complex was then transfected into HEK293T suspension adapted (HEK293Tsa) cells. Doxycycline and sodium butyrate was added 24 hours post-transfection (2 μg/mL and 5 mM, respectively) to induce transcription.

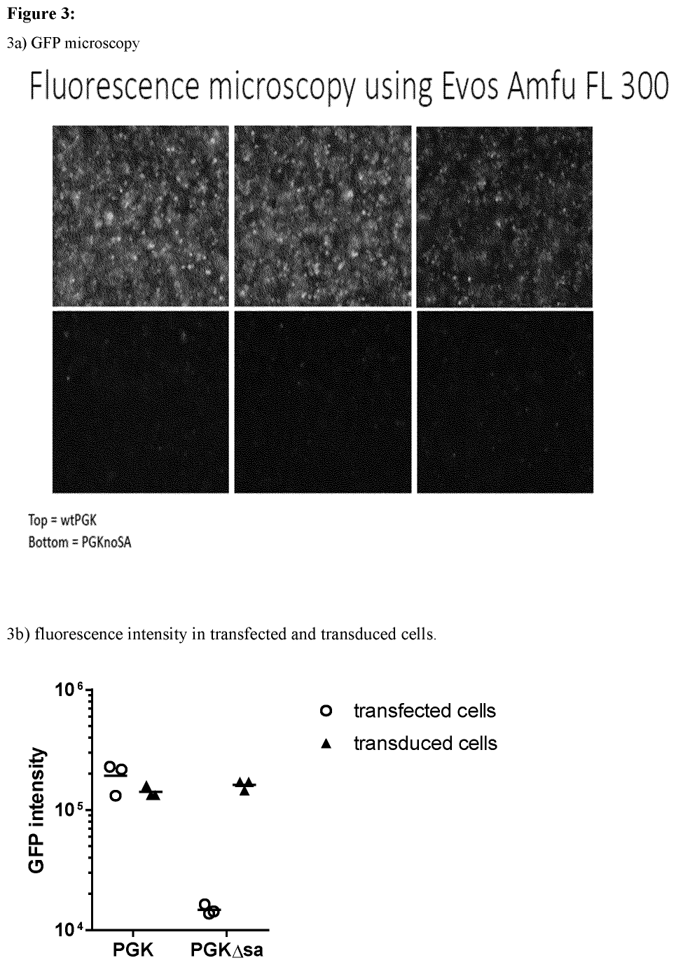

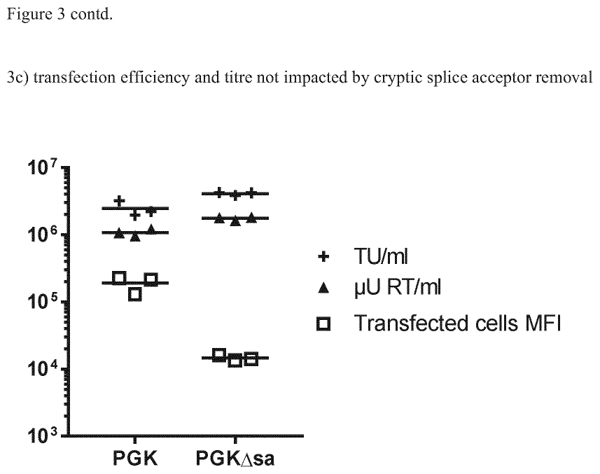

Two days post-transfection, cells were imaged for GFP protein using an Evos Amfu FL 300 inverted microscope (FIG. 3a). All images were captured using the same settings. As shown, GFP protein expression was lower in cells transfected with BmodT-PGKnoSA GW than BmodT-PGW.

In order to control for transfection efficiency, quantify the effect and assess for any impact on titre and transgene expression in transduced cells, the cells suspension was clarified by centrifugation at 500 g for three minutes. The vector-containing supernatant was collected and assayed for physical and functional titre, while the cell pellet was resuspended in 2% paraformaldehyde and analysed by flow cytometry. Since all cells were transfected with equal amounts of BAC1-pax, and physical titre is dependent on gag-pol expression but is independent of transfer plasmid, the physical titre was used to monitor transfection efficiency.

To determine functional titre, different volumes of vector-containing cell culture medium were applied to HEK293Tsa cells. Cells were subjected to flow cytometry analysis three days post-transduction to determine the percentage of cells expressing GFP. To assess whether removal of the cryptic splice acceptor impacted PGK promoter activity, the median fluorescence intensity of GFP+ cells was studied.

FIG. 3b shows the mean fluorescence intensity of all single cells within the transfected pool and the median fluorescence intensity of GFP+ transduced cells. The mean fluorescence intensity of all single cells was selected in transfected cells to avoid excluding weak GFP-expressing cells from any GFP+ gate.

FIG. 3c shows effect on transfection efficiency and titre of cryptic splice acceptor removal. Both functional and physical titre was similar for both vectors (PGK and PGKnoSA) indicating that removal of the cryptic splice acceptor had no impact on vector production or vector infectivity, while the fluorescence intensity of transfected producer cells was over 10-fold lower with PGKnoSA than PGK.

Results and Conclusions:

Using this measure, a ˜13-fold reduction in average fluorescence intensity in transfected cells was observed using PGKnoSA versus PGK, while no impact on transgene expression in transduced cells was observed (FIGS. 3a-3c). This finding is consistent with the observation that transgene expression in producer cells is from the translation of spliced transcripts originating from the 5′LTR. Hence, the removal of this splice acceptor has reduced transgene expression in producer cells.

In contrast, transgene expression in transduced cells is driven entirely by the promoter within the transgene cassette (e.g. PGK). Importantly the data shown in FIG. 3b demonstrates that the AG dinucleotide at the 3′ terminus of the PGK promoter can be removed with no or negligible impact on PGK activity in transduced cells.

The results of experiments described above have demonstrated that the identification and subsequent removal of cryptic splice acceptors in a transgene cassette that could result in transgene expression driven by the hybrid LTR promoter (i.e. where the 5′ ORF in the spliced mRNA is that of the transgene) is indeed an effective strategy to reduce transgene expression in producer cells. This strategy is not limited to the PGK promoter, and could be applied to many other promoters as described herein.

### Example 3: Use of Splice Traps to Divert Splicing from the Transgene to a Minimal ORF

However, with respect to the strategy described in example 2: a) one must first identify a potential cryptic splice acceptor that could permit transgene expression from LTR derived transcripts and b) mutagenesis of sequences within a promoter sequence may also alter transgene expression in transduced cells, and this would need to be assessed for each promoter.

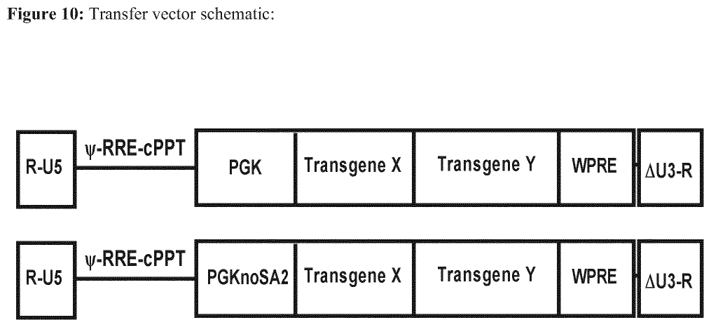

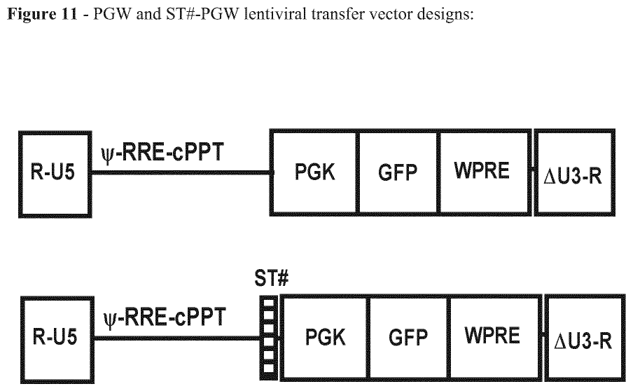

Therefore, an alternative strategy to reduce transgene expression in producer cells that overcomes these limitations was devised and this involves the use of “splice traps” since the majority of transgene expression in producer cells has been shown to be due to spliced transcripts originating from the LTR. These are short sequences that include a consensus splice acceptor site followed by a minimal open reading frame (ORF). We positioned these splice traps immediately upstream of the transgene cassette such that they are transcribed before the transgene cassette in LTR-derived mRNAs but excluded from transgene cassette derived mRNA. These sequences were designed to capture LTR-derived mRNAs that were destined to be spliced, thus diverting splicing away from the transgene cassette. A schematic showing layer of the elements present in the vectors used in example 3 is shown in FIG. 10. When used as a splice acceptor in conjunction with the major splice donor, the spliced mRNA would have a 5′ start codon belonging to the minimal ORF rather than the transgene. Minimal ORFs consist of a start codon followed by a stop codon. Since mammalian ribosomes utilise a 5′ ORF scanning mechanism for translational initiation and disassemble upon reaching a stop codon, downstream ORFs (e.g. transgene) are unlikely to be translated efficiently. Nevertheless, a second minimal ORF was positioned adjacent to the first, such that the ribosome must read through at least two start and stop codons prior to reaching transgene in any mRNA that has utilised the splice trap. Furthermore, to promote initiation at the first minimal ORF, a Kozak sequence was included to promote ribosomal initiation. Hence, efficient translation of transgene from such a processed mRNA is unlikely.

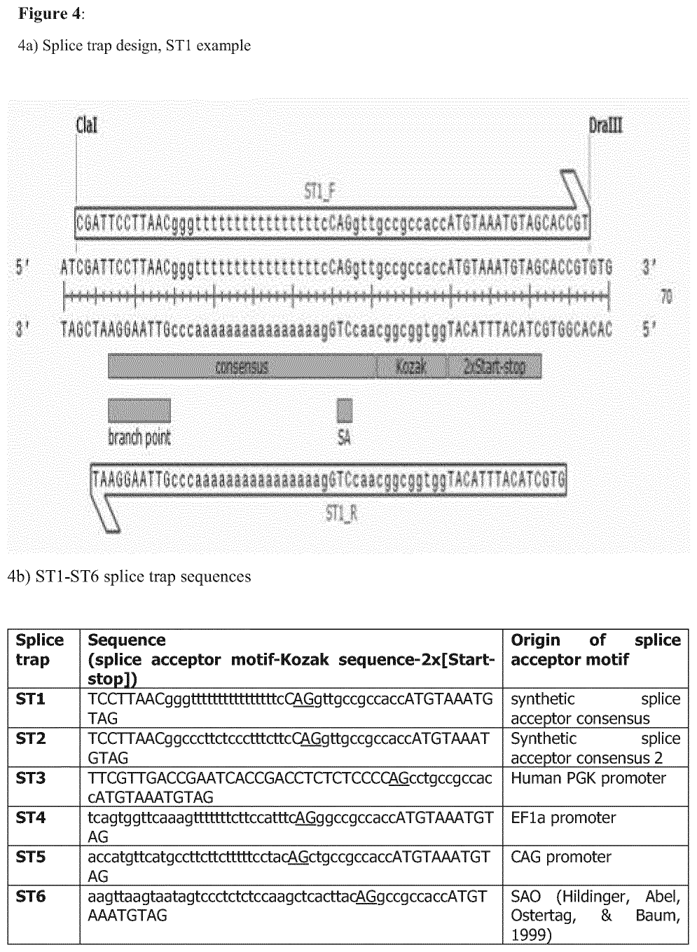

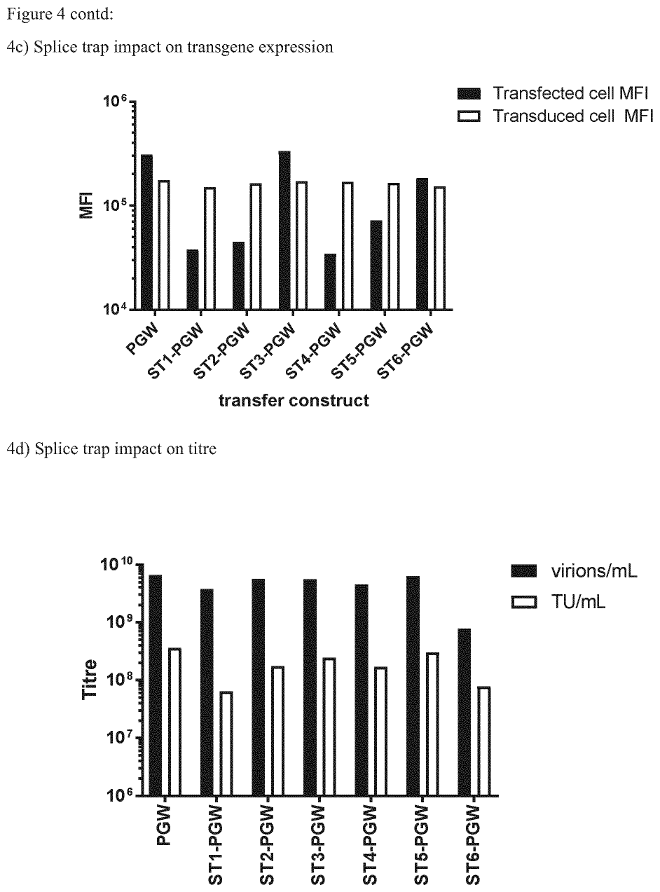

Two splice traps (ST1 and ST2) were designed based on a published splice acceptor consensus sequence. A third splice trap (ST3) used the splice acceptor identified within the PGK promoter. Splice traps 4 and 5 (ST4 and ST5) used well-known splice acceptors from the EF1α promoter or the rabbit beta-globin splice acceptor used in the CAG promoter, respectively. The final splice trap (ST6) used a splice acceptor sequence described to improve transgene expression in γ-retroviral vectors (Hildinger et al., 1999, Design of 5′ untranslated sequences in retroviral vectors developed for medical use. Journal of Virology, 73(5), 4083-4089). The sequence of these splice straps is shown in Table 2. All splice traps were assembled by annealing complementary oligonucleotides together (example shown in FIG. 4a) and cloned into BmodT-PGW between ClaI and DraIII restriction sites to generate BmodT-ST #-PGW. Construct identity was confirmed by restriction digest and Sanger sequencing. The resulting transfer plasmids were each co-transfected into HEK293Tsa cells with third generation packaging plasmids using PEIpro. As before, transfected cells were analysed for mean fluorescence intensity by flow cytometry while vector supernatant was assayed for physical and functional titre. Impact on transgene expression was assessed by measuring the median fluorescence intensity (MFI) of transduced cells. Results are shown in FIG. 4c.

As shown in FIG. 4c, none of the splice traps tested had an impact on PGK activity in transduced cells (shown in FIG. 4c as open bars). However transgene expression was found to be reduced by approximately 5-10 fold when splice traps 1, 2, 4 and 5 were present. Based on titre data (as shown in FIG. 4d), the transfection using ST6 appeared to be less efficient than other constructs, indicating that the ST6 MFI was underestimated. Hence, splice traps 3 and 6 were relatively ineffective at reducing transgene expression in producer cells. The difference in performance (as described above) between splice trap designs can be explained by their relative fit to the consensus splice acceptor. Their scores from MaxEntScan are shown in FIG. 5 and also in Table 4 below which also includes the human splice finder (HSF) score). As already described above, splice traps 3 and 6 score poorly, while the others have scored>12.

TABLE 4
showing scores from MaxEntScan for splice traps ST1-6

| | HSF 0-100 | MaxEntScan (3-13) |
|---|---|---|
| ST1 | 98.32 | 12.59 |
| ST2 | 95.64 | 12.54 |
| ST3 | 91.72 | 8.69 |
| ST4 | 93.08 | 12.38 |
| ST5 | 94.68 | 12.39 |
| ST6 | 89.15 | 3.16 |

From these results we suggest that a splice trap used in the invention preferably has a MaxEntScan score (performed as described in Yeo & Burge, J. Comput Biol. 2004; 11 (2-3) pp 377-94) greater than that of the splice acceptor in the promoter that leads to transgene expression in order to work optimally. Alternatively, the MaxEntScan of a splice trap used in the invention has a score greater than about 8.7. This is summarised in Table 5 below.

TABLE 5

| | | Splice trap |
|---|---|---|
| | MaxEntScan | activity upstream of |
| Splice trap | acceptor score | PGK-GFP-WPRE |
| St1 | 12.59 | High |
| St2 | 12.54 | High |
| St3 | 8.69 | Low |
| St4 | 12.38 | High |
| St5 | 12.39 | High |
| ST6 | 3.16 | Low |

In conclusion this data shows that splice traps can be used to effectively reduce transgene expression in producer cells, putatively by redirecting splicing from the major splice donor to the splice trap, which generates a mRNA that recruits the ribosome to a minimal ORF, rather than the transgene.

### Example 4: BAC Constructs Expressing Therapeutic Transgenes

Bacterial artificial chromosome (BAC) DNA constructs were engineered in which all vector components (gagpol, VSVg, rev, and transfer vector) that are required for producing lentiviral vectors and a zeocin resistance marker are expressed from a single large DNA construct. As the vector packaging components are controlled by tetracycline inducible promoters, production of the viral particles can be induced by the addition of doxycycline or tetracycline in culture. The transfer vector sequence contains transgenes encoding both (i) a chimeric antigen receptor (CAR) (designated Gene X) also (ii) a membrane protein (designated Gene Y), these are immediately 3′ to the wild type PGK promoter (wtPGK) or the PGK promoter modified to delete its ability to function as a splice acceptor here designated PGKnoSA2 (SEQ ID NO 19) (Note that PGKnoSA2 is the same as PGKnoSA described herein except that in PGKnoSA2 only 3′G is deleted whereas in PGKnoSA both 3′AG are deleted with respect to the wild type PGK promoter as shown in SEQ ID Nos 19-21). The layout of the elements in the transfer vector is shown in FIG. 10.

These BAC DNA constructs were transfected into suspension-adapted HEK293T cells using PEI based transfection reagents (e.g. PEIpro™). After 2 days, the cells were cultured in media supplemented with zeocin. Zeocin-resistant stable pools were subsequently generated. Single cells in droplets were deposited into 96-well plates containing growth media. Single cell derived colonies were formed after 19-21 days in culture.

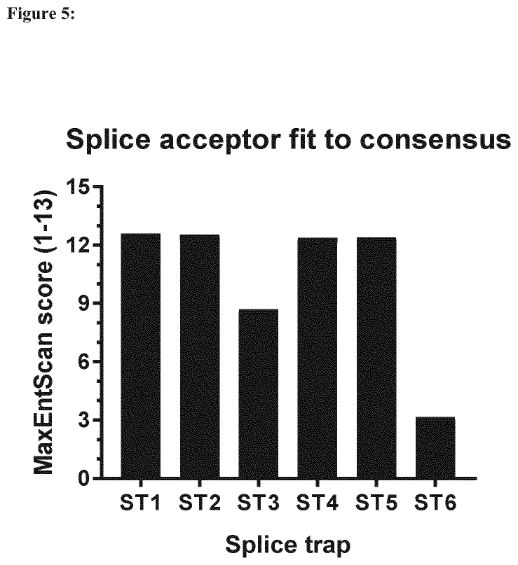

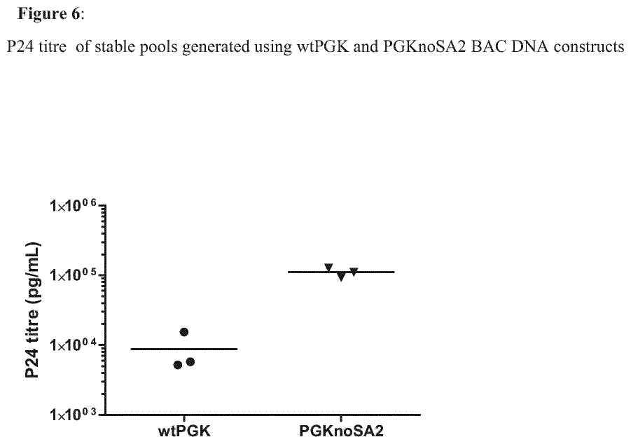

3 stable pools (SP1-3) were generated by transfecting BAC DNA constructs expressing the transgene (Genes x and Y) under the control of wtPGK or PGKnoSA2 promoter into HEK293T cells and selecting for resistance to zeocin. Lentivirus production was induced, supernatant was harvested and the titre of p24 (pg/mL) of supernatants were measured. Following induction of lenviral production with doxycycline, stable pools generated with the wtPGK construct produced significantly lower amounts of lentiviral vector (pg/mL p24) than that with the PGKnoSA2 construct (FIG. 6). The average p24 titres for the 3 stable pools were 8,803 and 111,000 for wtPGK or PGKnoSA2, respectively, representing a difference of 12.56 fold (FIG. 6).

Following stable pool generation single cells were deposited into 24 plates of 96 wells per plate for each of wtPGK stable pool 3 and PGKnoSA2 stable pool 2. Single cell derived colonies were formed after 3 weeks in culture (Colonies formed). These colonies were then picked and further expanded to a minimal of 4 mL cultures in the presence of zeocin selection (Clones grown up). Lentivirus production was induced for each clone, supernatant was harvested. Titre of p24 (pg/mL) was determined and the number of clones with >1E4 pg/mL was counted. TU/mL of supernatants was measured by quantifying the number of transduction units in transduced SupT1 cells, and the number of clones with >1E7 TU/mL were counted.

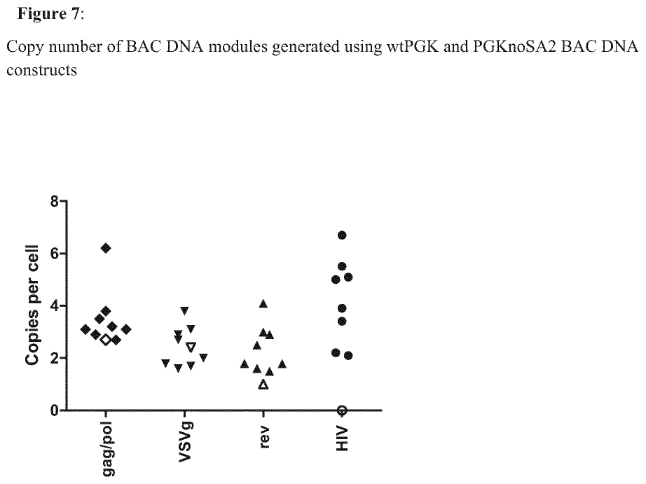

Of the cells transfected with the wtPGK construct, only 24 single cell-derived colonies were formed in the 96 well plates and 11 of those were successfully expanded in suspension culture to ≥4 mL scale. Following induction with doxycycline, >1E4 pg/mL p24 was detected in the supernatant of 2 of the 11 clones. Infectious titre was not detected even in the clone with the highest p24 titre (Table 6) and the transfer vector sequence was not present in this clone (FIG. 7).

TABLE 6
Number of clones generated using wtPGK
and PGKnoSA2 BAC DNA constructs

| | wtPGK | PGKnoSA2 |
|---|---|---|
| Number of wells printed | 2304 | 2304 |
| Single-cell derived colonies formed | 24 | 144 |
| Clones that managed to scale up to ≥4 mL | 11 | 89 |
| with zeocin | | |
| Clones with p24 titre >1E4 pg/mL | 2 | 36 |
| Clones with SupT1 ddPCR titre >1E7 TU/mL | 0 | 19 |

In contrast, of the cells transfected with the PGKnoSA2 construct, 144 single cell-derived colonies were formed in the 96 well plates and 89 of those were successfully expanded in suspension culture to ≥4 mL scale. Following induction with doxycycline, >1E4 pg/mL p24 was detected in the supernatant of 36 clones, >1E7 TU/mL, infectious titre was detected in 19 clones with higher p24 titres (Table 6) and the transfer vector sequence was present in these clones (FIG. 7).

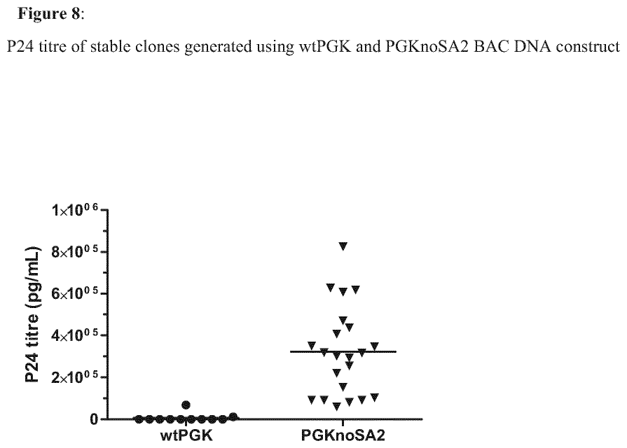

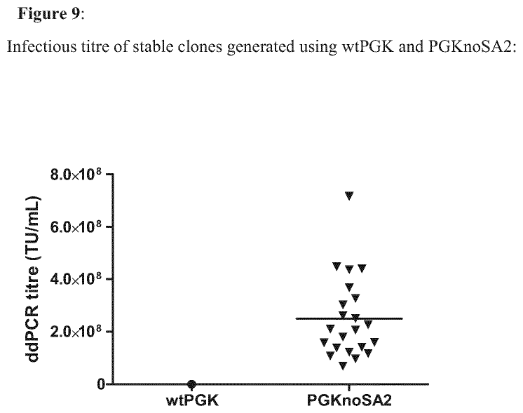

Only low amounts of lentivirus particles (pg/mL p24) were present in clones derived from wtPGK construct following induction, but high amounts of lentiviral particles were present in many of the PGKnoSA2 clones following induction (FIG. 8). No lentivirus infectious titre was detected in the clone derived from wtPGK construct following induction, but high infectious titres were present in many of PGKnoSA2 clones following induction (FIG. 9).

In summary, we found our therapeutic transgene cassette encoding Genes X and Y to be cytotoxic to producer cells when constitutively expressed such that high titre producer cell lines could not be generated. This problem was resolved by removing the splice acceptor in PGK (PGKnoSA2) which enabled the generation of stable cell lines capable of producing high titre lentiviral vector.

SEQUENCE LISTING
SEQ ID NO: 1:
Nucleotide sequence of splice acceptor
location (from 5′ boundary) of PGK-492 promoter:
TCACCGACCTCTCTCCCCAG
SEQ ID NO: 2:
Nucleotide sequence of splice acceptor
location (from 5′ boundary) of EFS (EFla short)-
184 promoter:
CGCAACGGGTTTGCCGCCAGAAC
SEQ ID NO: 3:
Nucleotide sequence of Promoter and spice acceptor
location (from 5′ boundary) of Ube-902
promoter:
TAGGCTTTTCTCCGTCGCAGGAC
SEQ ID NO: 4:
Nucleotide sequence of splice acceptor
location (from 5′ boundary) of Ube-1134 promoter:
GGTCAATATGTAATTTTCAGTGT
SEQ ID NO: 5:
Nucleotide sequence of splice acceptor
location (from 5′ boundary) of Ubc-TT89 promoter:
GTTTTTGGCTTTTTTGTTAGAC
SEQ ID NO: 6:
Nucleotide sequence of splice acceptor
location (from 5′ boundary) of SFFV-37T promoter:
CCCCTCACTCGGCGCGCCAGTCC
SEQUENCE LISTING contd:
SEQ ID NO: 7:
Nucleotide sequence of splice acceptor
location (from 5′ boundary) of SFFV-374 promoter:
GCGCGCCAGTCCTCCGACAGACT
SEQ ID NO: 8:
Nucleotide sequence of splice acceptor
location (from 5′ boundary) of SV40-T7T promoter:
CCGCCCCTAACTCCGCCCAGTTC
SEQ ID NO: 9:
Nucleotide sequence of splice acceptor
location (from 5′ boundary) of SV40-229 promoter:
AATTTTTTTTATTTATGCAGAGG
SEQ ID NO: 10:
Nucleotide sequence of splice acceptor
location (from 5′ boundary) of CMV-252 promoter:
GGGACTTTCCTACTTGGCAGTAC
SEQ ID NO: 11:
Nucleotide sequence of consensus sequence
around active splice acceptor in PGK promoter
TCACCGACCTCTCTCCCCAG(cct)
SEQ ID NO: 12:
Nucleotide sequence of consensus sequence
around active splice acceptor in modified PGK
promoter
TCACCGACCTCTCCCCgAG(cct)
SEQ ID NO: 13:
Nucleotide sequence of splice trap ST1
TCCTTAACgggttttttttttttttttcCAGgttgccgcc
accATGTAAATGTAG
SEQ ID NO: 14:
Nucleotide sequence of splice trap ST2
TCCTTAACggcccttctccctttcttcCAGgttgccgcca
ccATGTAAATGTAG
SEQ ID NO: 15:
Nucleotide sequence of splice trap ST3
TTCGTTGACCGAATCACCGACCTCTCTCCCCAGcctgccg
ccaccATGTAAATGTAG
SEQ ID NO: 16:
Nucleotide sequence of splice trap ST4
TcagtggttcaaagtttttttcttccatttcAGggccgcc
accATGTAAATGTAG
SEQ ID NO: 17:
Nucleotide sequence of splice trap ST5
AccatgttcatgccttcttctttttcctacAGctgccgc
caccATGTAAATGTAG
SEQ ID NO: 18:
Nucleotide sequence of splice trap ST5
aagttaagtaatagtccctctctccaagctcacttacAG
gccgccaccATGTAAATGTAG
SEQ ID NO: 19:
Nucleotide sequence encoding modified PGK promoter
in which the guanine in the active splice
site is deleted (PGKnoSA2)
Ggggttggggttgcgccttttccaaggcagccctgggttt
gcgcagggacgcggctgctctgggcgtggttccgggaaac
gcagcggcgccgaccctgggtctcgcacattcttcacgtc
cgttcgcagcgtcacccggatcttcgccgctacccttgtg
ggccccccggcgacgcttcctgctccgcccctaagtcggg
aaggttccttgcggttcgcggcgtgccggacgtgacaaac
ggaagccgcacgtctcactagtaccctcgcagacggacag
cgccagggagcaatggcagcgcgccgaccgcgatgggctg
tggccaatagcggctgctcagcggggcgcgccgagagcag
cggccgggaaggggcggtgcgggaggcggggtgtggggcg
gtagtgtgggccctgttcctgcccgcgcggtgttccgcat
tctgcaagcctcoggagcgcacgtcggcagtcggctccct
cgttgaccgaatcaccgacctctctcccca
SEQ ID NO: 20:
Nucleotide sequence encoding modified PGK promoter
in which the 3′ adenine and guanine in
the active splice site are deleted (PGKnoSA)
Ggggttggggttgcgccttttccaaggcagccctgggttt
gcgcagggacgcggctgctctgggcgtggttccgggaaac
gcagcggcgccgaccctgggtctcgcacattcttcacgtc
cgttcgcagcgtcacccggatcttcgccgctacccttgtg
ggccccccggcgacgcttcctgctccgcccctaagtcggg
aaggttccttgcggttcgcggcgtgccggacgtgacaaac
ggaagccgcacgtctcactagtaccctcgcagacggacag
cgccagggagcaatggcagcgcgccgaccgcgatgggctg
tggccaatagcggctgctcagcggggcgcgccgagagcag
cggccgggaaggggcggtgcgggaggcggggtgtggggcg
gtagtgtgggccctgttcctgcccgcgcggtgttccgcat
tctgcaagcctccggagcgcacgtcggcagtcggctccct
cgttgaccgaatcaccgacctctctcccc
SEQ ID NO: 21:
Nucleotide sequence encoding wild type PGK promoter
in which both the 3′ adenine and
guanine in the active splice site are
retained (highlighted in bold)
Ggggttggggttgcgccttttccaaggcagccctgggttt
gcgcagggacgcggctgctctgggcgtggttccgggaaac
gcagcggcgccgaccctgggtctcgcacattcttcacgtc
cgttcgcagcgtcacccggatcttcgccgctacccttgtg
ggccccccggcgacgcttcctgctccgcccctaagtcggg
aaggttccttgcggttcgcggcgtgccggacgtgacaaac
ggaagccgcacgtctcactagtaccctcgcagacggacag
cgccagggagcaatggcagcgcgccgaccgcgatgggctg
tggccaatagcggctgctcagcggggcgcgccgagagcag
cggccgggaaggggcggtgcgggaggcggggtgtggggcg
gtagtgtgggccctgttcctgcccgcgcggtgttccgcat
tctgcaagcctccggagcgcacgtcggcagtcggctccct
cgttgaccgaatcaccgacctctctccccag

1. A nucleic acid module encoding at least one transgene, wherein said module comprises
(a) a first promoter which is operably linked to retroviral nucleic acid sequences encoding:
(i) cis-acting retroviral elements such as LTRs,
(ii) a retrovirus leader sequence comprising a splice donor site, and (b) a second promoter for transgene expression operably linked to said transgene and wherein said second promoter for transgene expression is modified such that it comprises (a) an attenuated splice acceptor site; and/or (b) said second promoter has a splice trap sequence positioned upstream and/or downstream of said promoter which reduces or eliminates splicing at its position.

2. A module according to claim 1, wherein said splice acceptor site is attenuated by at least one modification of said site selected from: (i) a total deletion (ii) partial deletion, (iii) an insertion (iv) a substitution.

3. A module according to claim 1, wherein said retroviral nucleic acid sequences encode gag, pol and env proteins (or functional equivalents thereof) and also a non-mammalian origin of replication.

4. A module according to claim 1, wherein said retroviral nucleic acid is derived from a lentivirus, alpha-retrovirus, gamma-retrovirus or foamy-retrovirus.

5. A module according to claim 1, wherein said retroviral nucleic acid is derived from a lentivirus.

6. A module according to claim 5, wherein said lentivirus is selected from: HIV-1, HIV-2, SIV, FIV, EIAV and Visna.

7. A module according to claim 1, which comprises a splice trap sequence and wherein said splice trap sequence is selected from any such sequence that has a MaxEntScan splice acceptor score that is greater than 9.

8. A module according to claim 7, which comprises a splice trap sequence and wherein said splice trap sequence is selected from any one of the splice traps designated ST1, ST2, ST4 or ST5 or a functional fragment thereof.

9. A module according to claim 8, which comprises a splice trap sequence and wherein said sequence is followed by a minimal open reading frame (ORF) which is less than about 50 codons.

10. A module according to claim 1, which comprises a splice trap sequence and wherein said splice trap sequence is in a position selected from: (i) immediately upstream of the transgene, (ii) between a major splice donor site (MSD) and transgene cassettes transcriptional start site, or (iii) between a MSD and polyA.

11. A module according to claim 1, wherein said first promoter is CMV or RSV and said second promoter is selected from SV-40, PGK, EFS, SFFV, Ubc operably linked to the transgene.

12. A module according to claim 1, wherein said transgene is a therapeutic transgene.

13. A module according to claim 11, wherein said retroviral nucleic acid is lentiviral nucleic acid derived from HIV-1 or HIV-2 and which further comprises a second promoter which is a human PGK promoter having an attenuated splice acceptor site and/or a splice trap operably linked to the transgene.

14. A module according to claim 11, wherein said PGK promoter is modified such that it comprises a wholly or partially deleted splice acceptor site and wherein said splice acceptor site is located at the position of the last two base pairs adenine and guanine of said PGK promoter sequence.

15. A module according to claim 14, wherein said modified PGK promoter has the nucleic acid sequence shown in SEQ ID NO 19 or 20.

16. A vector comprising a nucleic acid module according to claim 1.

17. A vector according to claim 16, which is a plasmid.

18. A vector, which is a bacterial artificial chromosome (BAC) which can hold at least 25 kb of DNA and which comprises a nucleic acid module according to claim 3.

19. A cell comprising the nucleic acid module of claim 1.

20. A cell according to claim 19, which is a mammalian cell.

21. A method of producing a replication defective retroviral vector particle expressing a therapeutic transgene, comprising the following steps:
(a) introducing the nucleic acid module of claim 3, into a culture of mammalian host cells; and
(b) culturing the mammalian host cells under conditions in which the replication defective retroviral vector particle is produced, and then
(c) isolating said replication defective retroviral vector particles.

22. A method of producing a replication defective retroviral vector particle expressing a therapeutic transgene comprising the following steps:
(a) introducing (i) the nucleic acid module of claim 1 into culture of mammalian cells and also (ii) one or more further nucleic acid modules encoding gag, pol and env retroviral sequences and also a non-mammalian origin of replication into said cells; and
(b) culturing the mammalian host cells under conditions in which the replication defective retroviral vector particle is produced, and then
(c) isolating said replication defective retroviral vector particles.

23. A replication defective retrovirus obtained or obtainable by the method of claim 21.

24. A replication defective retrovirus according to claim 23, which is a lentivirus expressing a therapeutic transgene.

25. A replication defective retrovirus according to claim 23, which comprises the PGK promoter with an attenuated splice acceptor site and/or a splice trap operably linked to said therapeutic transgene.

26. A replication defective retrovirus according to claim 23, which comprises a splice trap selected from any one of the splice traps designated ST1, ST2, ST4 or ST5, or a functional fragment thereof.

27. A pharmaceutical composition comprising a replication defective retrovirus according to claim 23 and a pharmaceutically acceptable excipient.

28.-29. (canceled)

30. A method of genetically modifying mammalian cells in vitro comprising (i) introducing a replication defective retrovirus according to claim 23 into said cells and then (ii) culturing said cells under conditions suitable for growth.

31. A method according to claim 30, wherein said cells are human cells.

32. A method according to claim 31, wherein said human cells are autologous cells.

33. Cells obtained or obtainable by the method of claim 30.

34. A pharmaceutical composition comprising cells according to claim 30 in combination with a pharmaceutically acceptable excipient.

35. A method of treating disease in a human subject in need thereof comprising administering to said subject a replication defective retrovirus according to claim 23.
